(19) United States
(12) Patent Application Publication
(10) Pub. No.: US 2002/0065224 A1
(43) Pub. Date: May 30, 2002

(54) Caveolin-1 gene and polypeptide encoded thereby and methods of use thereof

(76) Inventors: Florent C. Bender, Epalinges (CH); Marc A. Reymond, Epalinges (CH); Claude Bron, Epalinges (CH); Andrew Quest, Epalinges (CH)

Correspondence Address: MINTZ, LEVIN, COHN, FERRIS,, GLOVSKY and POPEO, P.C., One Financial Center, Boston, MA, 02111, US

(21) Appl. No.: 09/976,773

(22) Filed: Oct. 12, 2001

Related U.S. Application Data
(60) Provisional application No. 60/242,545, filed on Oct. 23, 2000.

(30) Foreign Application Priority Data
Oct. 13, 2000 (DE) 100 53 047.8

(51) Int. Cl. A61K 38/17; A61K 48/00; G01N 33/574

(52) U.S. Cl. 514/12; 514/44; 424/93.2; 435/7.23

(57) ABSTRACT

The invention relates to compositions comprising caveolin polypeptides and nucleic acids, and methods of use thereof. The invention is useful in the treatment of non-steroid dependent carcinoma, especially for treatment of gastrointestinal carcinoma. According to the invention, caveolin-1 or the gene encoding caveolin are especially preferred to treat colon carcinoma.

### RELATED APPLICATION

This application claims priority to German Patent Application No. 100 53 047.8, filed Oct. 13, 2000, and to U.S. Ser. No. 60/242,545, filed Oct. 23, 2000, which are incorporated herein by reference in their entireties.

### BACKGROUND OF INVENTION

During progression from a normal epithelium to invasive or metastatic cancer, cells accumulate a combination of genetic mutations, including activation of oncogenes including ras and myc, as well as inactivation of tumor-suppressor genes such as p53 and RB. As a general consequence, several signal transduction pathways become constitutively activated. This activation leads to aberrant cell proliferation, loss of adhesion and a transformed phenotype coupled with insensitivity to apoptosis.

Caveolin-1 has been implicated in normal cell proliferation and cell transformation. Caveolin-1 mRNA and protein levels are reduced in transformed and tumor cell lines, suggesting that reduced caveolin-1 expression may represent a general characteristic of transformed cells, and that caveolin-1 may be an inhibitor of tumor induction and/or progression.

Caveolin-1 is part of a multi-gene family including caveolin-1, caveolin-2 and caveolin-3. Caveolin-1 is a 21-kDa coat/adapter protein of caveolae. Caveolin-1 has a scaffolding domain thought to interact with proteins involved in several signal transduction pathways, e.g. heterotrimeric G proteins, Ha-Ras, c-Src, eNOS, PKCα, MAPK and tyrosine kinase receptors (See e.g., Li et al., J. Biol. Chem. 271:29182-90, 1996). Many of these proteins contain a consensus motif for caveolin-1 binding (See Anderson, Annu. Rev. Biochem. 67:199-255, 1998). The human caveolin-1 gene is known (See Engelmann et al., FEBS letters 436:403-410, 1998). The function of caveolin-1 in human cancers is unclear. Some reports suggest that caveolin-1 functions as a tumor suppressor protein in the NIH-3T3 mouse fibroblast cell line, human breast cancer cell lines and lung carcinoma cell lines (See Koleske et al., Proc. Natl. Acad. Sci. USA 92 (1995), 1381-1385; Lee, S. W. et al., Oncogene 16 (1998), 1391-1397; and Racine C. et al., Biochem. Biophys. Res. Commun. 255 (1999), 580-586). U.S. Pat. Nos. 5,783,182 and 6,252,051 disclose that caveolin sequences can be used to identify and target metastatic cells, such as metastatic prostate cancer cells. However, no mutations in caveolin-1 have been detected in human cancer cells. In addition, CpG islands associated with the caveolin-1 gene are methylated in either primary tumors or tumors-derived cell lines (see Prostate. 2001 Feb 15;46(3):249-56; FEBS Lett. 1999 Apr 9;448(2-3):221-30.), though this issue is still controversial (see Oncogene. 1999 Mar 11;18(10):1881-90.).

Caveolin-1 has been found to function as a tumor suppressor in human non-steroid dependent carcinoma, especially in gastrointestinal carcinoma. Further, it was found that caveolin-1 re-expression in human non-steroid dependent carcinoma cells reduces their ability to form tumors. It was also found that 1) caveolin-1 protein levels were reduced in colon tumors from human patients; 2) colon carcinoma cells had low levels of caveolin-1 mRNA and protein; 3) expression of caveolin-1 in the colon carcinoma lines HT-29 and DLD-1 blocked or retarded tumor formation in nude mice; and 4) the ability of HT29-cav- 1 to form tumors in nude mice, despite initial caveolin-1 presence, was linked to a selection process favoring proliferation of those cells with reduced basal caveolin-1 levels.

### SUMMARY OF INVENTION

The invention generally relates to the use of a therapeutically effective amount of a caveolin protein or a caveolin gene. While caveolin-1 is used as a specific, non-limiting example, it would be obvious to one skilled in the art to modify the teachings of the present invention for the use of caveolin-2, caveolin-3, and other caveolin family members.

One aspect of the present invention relates to a method of treatment of a cell proliferation-associated disorder, e.g. cancer, using a therapeutically effective amount of a caveolin-1 polypeptide. In one embodiment, the cancer is non-steroid dependent carcinoma, e.g. gastrointestinal carcinoma. In another embodiment, a caveolin-1 polypeptide is especially preferred to treat colon carcinoma or stomach carcinoma.

A second aspect of the present invention relates to a method of treatment of a cell proliferation-associated disorder, e.g. cancer, using a therapeutically effective amount of a caveolin-1 nucleic acid. In a related embodiment, the cancer is non-steroid dependent carcinoma, e.g. gastrointestinal carcinoma. In another embodiment, a caveolin-1 nucleic acid is especially preferred to treat colon carcinoma or stomach carcinoma.

In preferred embodiments, a caveolin-1 polypeptide or nucleic acid is provided on a delivery vehicle. According to the invention, delivery vehicles may be antibodies such as monoclonal antibodies, which specifically bind to an antigen related to a polypeptide present on a cancer cell, e.g. a non-steroid dependent carcinoma. Other delivery vehicles according to the invention include liposomes; vectors, particularly viral vectors; and particles made of a chemically inert substance, e.g. gold or diamond.

A third aspect of the present invention relates to methods for identifying a potential therapeutic agent for use in treatments of a caveolin-associated pathology, e.g. cancer, by providing a cell that expresses a caveolin-1 polypeptide such that a property or function that can be ascribed to the polypeptide is present in the cell, then contacting the cell with a potential therapeutic agent, then determining that the agent alters the property or function of the cell if the alteration occurs in the presence but not in the absence of the agent.

A fourth aspect of the invention relates to methods for identifying a carcinoma, e.g. a non-steroid dependent carcinoma, in a subject, such as by providing a test cell population from the subject, measuring the amount of caveolin-1 nucleic acid expressed in at least one cell of the test cell population, comparing the amount of caveolin-1 nucleic acid in the test cell population with a reference cell population whose carcinoma stage is known, then identifying a difference in expression levels between the two populations. Carcinoma stage is defined here as the presence, absence or extent of carcinoma in a cell, tissue, organ, or organism.

A fifth aspect of the invention relates to methods for assessing the efficacy of treatment of a carcinoma in a subject, such as by providing a by providing a test cell population from the subject, measuring the amount of caveolin-1 nucleic acid expressed in at least one cell of the test cell population, comparing the amount of caveolin-1 nucleic acid in the test cell population with a reference cell population whose carcinoma stage is known, then identifying a difference in expression levels between the two populations.

A sixth aspect of the invention relates to methods for identifying cancerous tissue, such as by contacting a test tissue at risk for or affected by cancer with an analyate, e.g. an antibody, capable of recognizing a caveolin moiety, e.g. a caveolin-1 polypeptide, quantifying the analyate binding to the test tissue, then comparing the binding of the analyate to the test tissue with the binding of the analyate to a reference tissue whose carcinoma stage is known to identify cancerous tissue. Generally, an analyate is any physical, chemical, biochemical or biological substance capable of being analyzed. By way of non-limiting example, an analyate is a molecule, a drug, a small molecule, a macromolecule, a polymer, an amino acid, a protein, an antibody, a protein complex, a polysaccharide, a nucleic acid, a particle, an inert material, an organelle, a cell, a microorganism, a bacteria, a virus, a fungus, a prion, a tumor, a tissue, a cellular environment comprising cancerous tissue, a cellular environment comprising diseased tissue, or a wound.

In a first related embodiment, the analyate is an antibody and the caveolin moiety is a polypeptide. In second related embodiment, the analyate is a first nucleic acid and the caveolin moiety is a second nucleic acid. In a related aspect, this second nucleic acid can be genomic DNA, mRNA or eDNA.

A seventh aspect of the invention relates to a composition comprising a caveolin polypeptide and a pharmaceutically acceptable carrier.

An eighth aspect of the present invention relates to a composition comprising a caveolin nucleic acid and a pharmaceutically acceptable carrier.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DESCRIPTION OF THE FIGURES

FIG. 1 is a Northern blot analysis of normal human tissue samples and comparison with the colon carcinoma cell line, SW480. Multiple tissue or cell Northern blots (Clontech) containing equal amounts of poly (A)⁺RNA per lane were hybridized either with [³²P]labeled probes for caveolin-1 (upper panel) or β-actin (lower panel), as a control. Heart and peripheral blood leukocytes (PBL) represent positive and negative controls for caveolin-1 expression, respectively. Migration of RNA markers (kb) is indicated to the left of the panel.

FIG. 2 shows an analysis of caveolin-1 expression in several human colon carcinoma cell lines. In FIG. 2A, lysates from the indicated cell lines were prepared as described and analyzed by Western blotting. Proteins from carcinoma cells (50 μg) or MDCK cells (5 μg) were separated by SDS-PAGE, transferred to nitrocellulose and subsequently either caveolin-1 (upper panel) or actin (lower panel) were detected using specific antibodies. Migration positions of marker proteins are indicated to the left (kDa). In FIG. 2B, Northern blot analysis of caveolin-1 mRNA expression. Samples containing cytoplasmic RNA from carcinoma cells (20 μg) or MDCK cells (2 μg) were separated on a 1% agarose gel, transferred to nylon membrane and probed for caveolin-1 as described. Cells from which RNA was prepared are indicated at the top. To the left, the position of the 28S and 18S ribosomal RNA are indicated.

FIG. 3 shows caveolin-1 protein expression in colon tissues from four patients (G009, G010, G011 and G017) with colon cancer. Tissues were excised by surgery from normal and tumor sites, and colon mucosa was separated from the rest of the stroma by affinity purification as described. Proteins from lysates of the indicated tissues (10 μg) or from MDCK cells (5 μg), were treated as described in FIG. 2. Loading in individual lanes was controlled by Ponceau Red S staining after transfer to nitrocellulose. Abbreviations used are: NM, normal mucosa; TM; tumor mucosa; NS, normal stroma; TS, tumor stroma; M, MDCK cells.

FIG. 4 shows caveolin-1 expression in NIH-3T3 cells after tumor formation in nude mice. FIG. 4A shows lysates from MDCK cells, parental NIH-3T3 cells or NIH-3T3 cells obtained upon tumor formation in nude mice (ExTumor), which were prepared as described. Proteins from NIH-3T3 cells (50 μg) or MDCK cells (5 μg) were treated as in FIG. 2A. FIG. 4B shows samples containing cytoplasmic RNA (15 μg) from MDCK cells, parental NIH-3T3 cells or NIH-3T3 ExTumor, which were analyzed by Northern blot analysis as described in FIG. 2B.

FIG. 5 shows IPTG-inducible expression of recombinant caveolin-1 in the human colon carcinoma cell lines HT29 and DLD1 . HT29 (A) or DLD1 (B) cells were stably transfected with caveolin-1 under the control of an IPTG-inducible promoter as described. After growth for 24 h in the absence (-) or presence (+) of 1 mM IPTG, cell lysates were prepared from transfected-(mock, C13, C14, C16) and parental HT29 cells (A), or from transfected- (mock, C2, C4) and parental DLD1 cells. Proteins (20 μg) were analyzed by Western blotting as in FIG. 2. Extracts of MDCK cells (5 μg total protein) were included as a positive control for caveolin-1 detection.

FIG. 6 shows tumor development in mice implanted with HT29 cells transfected with caveolin-1. 1×10⁶ cells were injected subcutaneously into 6-8 week old nude mice. A total of n=13 mice were analyzed in this fashion. For each mouse, control cells (parental HT29 cells or mock transfected cells) were injected on the left and HT29 cells transfected with caveolin-1 (clones C13, C14, C16) on the right. Large (D) and small (d) diameters of growing tumors were measured twice a week and corresponding volumes (V) were estimated using the equation V=d2×D×π/6. Results from a representative series of experiments with 4 mice are presented.

FIG. 7 shows tumor development in mice implanted with DLD1 cells transfected with caveolin-1. 1×10⁶ cells were injected subcutaneously into 6-8 week old nude mice. A total of n=7 mice were analyzed in the same fashion as described in FIG. 6. Results from a representative series of experiments with 4 mice are presented.

FIG. 8 shows an immunoblot analysis of caveolin-1 expression in transfected HT29 cells after tumor formation in nude mice. Tumors that developed upon injection of parental, mock or caveolin-1 transfected HT29 cells were excised as described and cultured (ExTumor). When homogenous cells populations were obtained, cells were lyzed and proteins (50 μg) analyzed by Western blot as in FIG. 2. Caveolin-1 expression in the absence or presence of IPTG was compared in samples from cells before (BI) and after (ExTumor) injection in mice. Results for cell populations obtained from two separate tumors (T1 and T2) are presented in each case.

FIG. 9 shows caveolin-1 expression in colon carcinoma cells resistant to high doses of methotrexate or with high metastatic potential. In FIG. 9A, expression of caveolin-1 is shown in stably differentiated HT-29 populations of the enterocytic (SM 12) or mucous-secreting (5M21) phenotype obtained by exposure of HT-29 cells to high concentrations of methotrexate was analyzed by Western blot analysis. Proteins from carcinoma (50 μg) or MDCK (5 μg) cells were analyzed as described in FIG. 2A. In FIG. 9B, expression of caveolin-1 protein in the colon carcinoma line Lovo and in two derived clones selected for high metastatic potential (E2 and C5) was analyzed by Western blot analysis as in A.

### DETAILED DESCRIPTION OF THE INVENTION

The present invention is based in part on the discovery of altered patterns of caveolin-1 expression in cancer as well as the tumor suppressor activity of caveolin-1 in human cancer. A caveolin-1 nucleic acid and polypeptide encoded thereby are disclosed in, e.g., Accession number NM_001753. The present invention encompasses any nucleic acid with greater than 95% identity to caveolin-1. Further, the present invention encompasses any polypeptide with 60% identity to caveolin-1, preferably greater than 80% identity to caveolin-1, even more preferably greater than 90% identity to caveolin-1 and still more preferably greater than 95% identity of caveolin-1. Alternative isoforms such as caveolin-1 beta and related genes, including but not limited to caveolin-2 (e.g. Accession number: NM_{—}001233), caveolin-3 (e.g. Accession number: XM_{—}052177), and other caveolins, are also encompassed by the present invention.

Several studies have recently assessed the potential role of caveolin-1 in tumor formation. When, for instance, caveolin-1 was re-expressed in breast cancer cell lines, cell proliferation in culture and anchorage-independent growth in soft agar were reduced compared to parental lines, suggesting that caveotin-1 modulates growth parameters generally considered relevant to tumor formation in vivo (8). However, no direct evidence was provided showing that presence of caveolin-1 prevented tumor formation. In a NIH-3T3 cell model system, re-expression of caveolin-1 in oncogenically transformed cells suppressed the transformed phenotype, since anchorage-independent growth in soft agar was abrogated (18). In addition, downregulation of caveolin-1 by overexpression of an anti-sense caveolin-1 construct was sufficient to mediate cell transformation and promote tumor formation when cells were injected in nude mice (19). Taken together, these results show that caveolin-1 can reduce cell tumorigenicity in the NIH-3T3 mouse fibroblast cell line and suggest it may do the same in human breast cancer cells.

Caveolin-1 was identified as one of 26 candidate tumor suppressor genes in human mammary carcinomas using differential display and subtractive techniques (31). In addition, the caveolin-1 gene has been mapped to a tumor suppressor locus in both the human (locus D7S522) and mouse (locus 6-A2/73 1) genomes (36, 37). These regions are frequently deleted or contain break point sites for chromosome translocation in a wide variety of tumors (36, 38). Furthermore, caveolin-1 was recently identified as a target protein for p53-dependent regulation (39). However, at the DNA level there is virtually no evidence that caveolin-1 is a tumor suppressor gene, since the caveolin-1 gene is neither mutated nor methylated in cancer cells (20), although methylation of the caveolin-1 promoter has been described in breast cancer cell lines and prostate cancer cells (40; see also Prostate. 2001 Feb 15;46(3):249-56.).

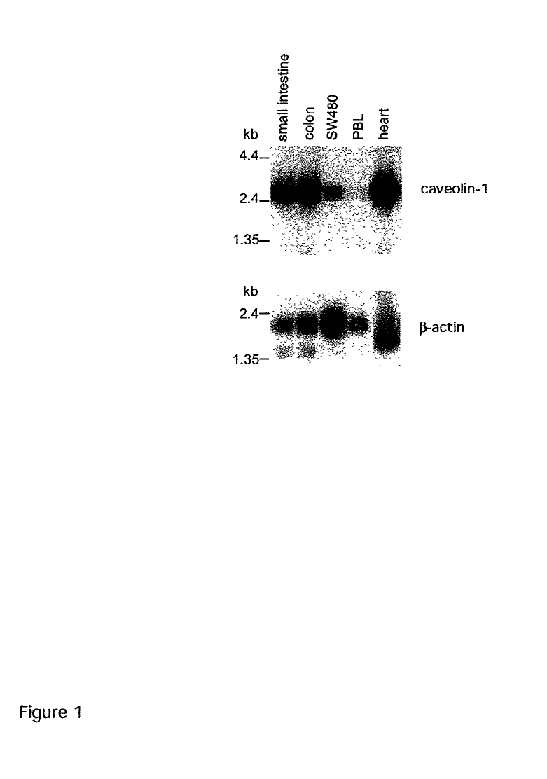

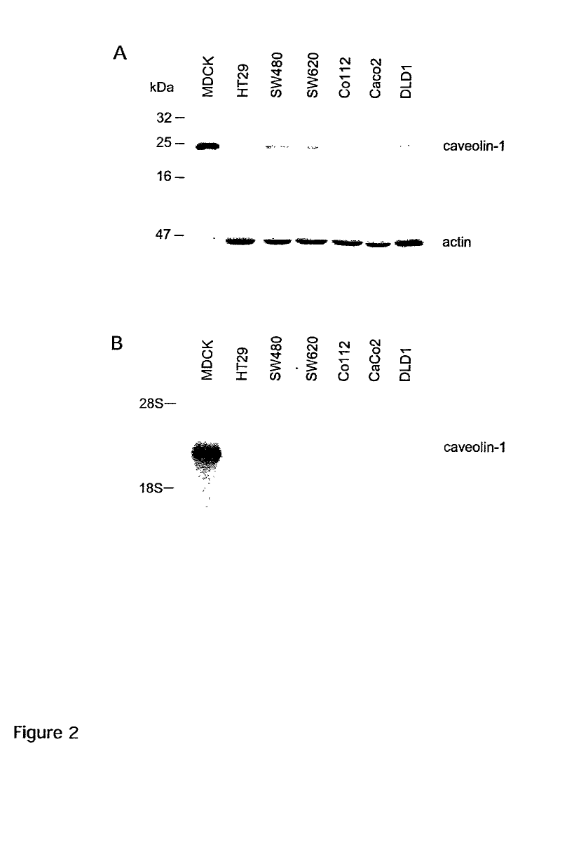

Caveolin-1 mRNA and protein levels (FIG. 1 and 2) are reduced in colon carcinoma cell lines as compared to normal colon tissue. Thus, after lung (9) and possibly breast (8), colon carcinomas represent a third group of human carcinomas where caveolin-1 levels are reduced as a consequence of what appears to be predominantly transcriptional regulation.

Moreover, the comparison of samples from normal colon and colon tumor tissue revealed that caveolin-1 protein expression was reduced in tumor epithelium, thereby establishing a direct link between reduced caveolin-1 expression levels observed in human colon carcinoma cell lines and a reduction of caveolin-1 expression observed in colonic epithelial cells upon tumor formation.

Caveolin-1 downregulation was not only observed in colon tumor mucosa, but also in the adjacent stroma, suggesting carcinoma cells may be able to modulate expression levels of caveolin-1 in surrounding tissues, mostly constituted of adipocytes, endothelial and muscle cells. Angiogenesis activators such a VEGF, bFGF, and HGF downregulate caveolin-1 in human endothelial cells (41).

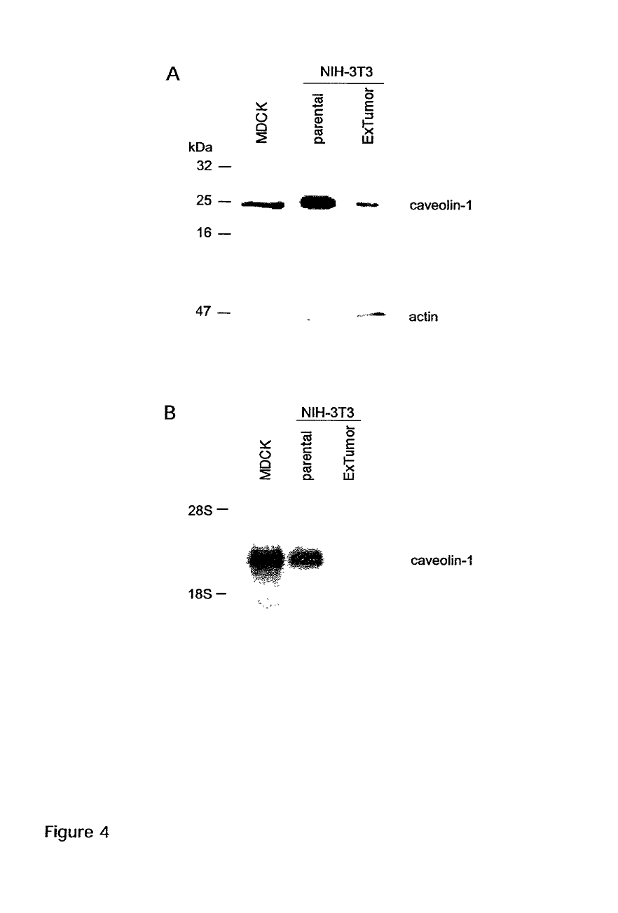

Tumors formed in nude mice yielded a cell population with less caveolin-1 (FIG. 4). Thus, caveolin-1 may be rate limiting for anchorage-independent growth and tumor formation in mice, similar to how NIH-3T3 ex-tumor cells with lower caveolin-1 levels form tumors more rapidly upon reinjection into nude mice.

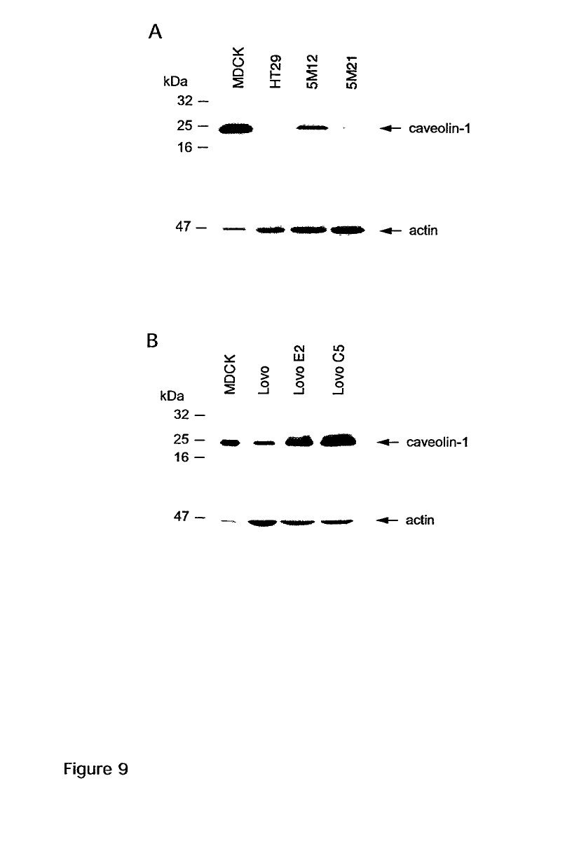

Caveolin-1 levels were highest in metastases derived from primary prostate tumors. Accumulation of caveolin- I relative to normal epithelium occurs with progression of prostate cancer (42). Caveolin-1 mRNA and protein are present at high levels in normal colon epithelium (FIGS. 1, 2 and 3), whereas only minimal expression is observed in the corresponding prostate tissue samples (42). Thus, transformation and progression of malignancy in cells that normally express caveolin-1 occurs in two phases: downregulation of caveolin-1 during primary tumor formation, then up-regulation of caveolin-1 occurs in methotrexate resistant HT29 cells (FIG. 9A), and multidrug-resistant human colon carcinoma HT-29 cells and breast carcinoma MCF-7 cells (44). Re-expression of caveolin-1 may also be required during metastasis.

Colon carcinoma clones selected from the Lovo line for higher metastatic potential (35) have elevated caveolin-1 protein levels when compared to parental cells (FIG. 9B). Basal caveolin-1 levels are higher in Lovo than other colon carcinoma lines (see FIG. 9B). While the cell populations E2 and C5 were obtained by sequential injection into mice followed by isolation of cells from resulting lung metastases, this does not require metastases to have higher levels of caveolin-1 expression than the original tumor. For instance, the primary colon tumor cells SW480 and matched metastatic colon cancer cells SW620, originating from the same patient, both have equally low caveolin-1 levels (FIG. 2). Similarly low caveolin-1 levels were also observed for liver (Isreco2) and peritoneal (Isreco3) metastases derived from a primary ascending human colon cancer (Isrecol), cell lines characterized by Sordat and co-workers (45). Taken together, this would argue that control of caveolin-1 levels in colon carcinomas is complex and that no simple unifying hypothesis is currently available to explain all available observations. Downregulation of caveolin-1 might be an early event that occurs in primary tumor formation of a limited set of epithelia that normally express high levels of caveolin-1, including colon and lung (9).

The precise mechanism by which reduced levels of caveolin-1 expression in epithelium would promote initial steps towards carcinoma formation is not clear. Several reports indicate that caveolin-1 possesses a specific motif, referred to as the scaffolding domain, which can bind to and inhibit the activity of a number of proteins involved in signal transduction, including heterotrimeric G proteins (11), Src family tyrosine kinases (10), endothelial nitric oxide synthase (eNOS) (46-49), Neu tyrosine kinase (50), EGF-receptor (51) and PKCα (52). Thus reduced levels of caveolin-1 would prolong cell stimulation linked to one of these numerous signal transduction pathways. Consistent with this notion, targeted downregulation of cavolin-1 in NIH-3T3 cells leads to hyperactivation of the p42/p44 MAP kinase pathway and as consequence cell transformation (19).

However, overexpression of caveolin-1 inhibited both MAPK-dependent and independent pathways in adipose cells, while in Cos-7 cells, caveolin-1 enhanced MAPK-dependent signaling (53). Thus, modulation of the MAPK, as well as other, signaling pathways by caveolin-1 may be differentially regulated depending the cell system studied. In addition, caveolin-1 levels are likely to be tightly controlled in cells since both up- and down-regulation alter cell signaling events.

Caveolin-1 expression has been reported to inhibit transcription of the cyclin D1 gene, suggesting that loss of caveolin-1 expression during tumorigenesis may lead to cellular transformation via the β-catenin/TCF/LEF signaling pathway (54, 55). Caveolin-1 is also involved in signal transduction events mediated by several integrins upon binding to extracellular matrix proteins. There, caveolin-1 plays a key role by linking integrins to Fyn activation, which in turn is responsible for Shc recruitment, regulation of Ras-MAP kinase signaling and cell cycle progression (56). Thus anchorage-independent growth, observed in transformed cells upon downregulation of caveolin-1, may be linked to this particular aspect of caveolin-1 function.

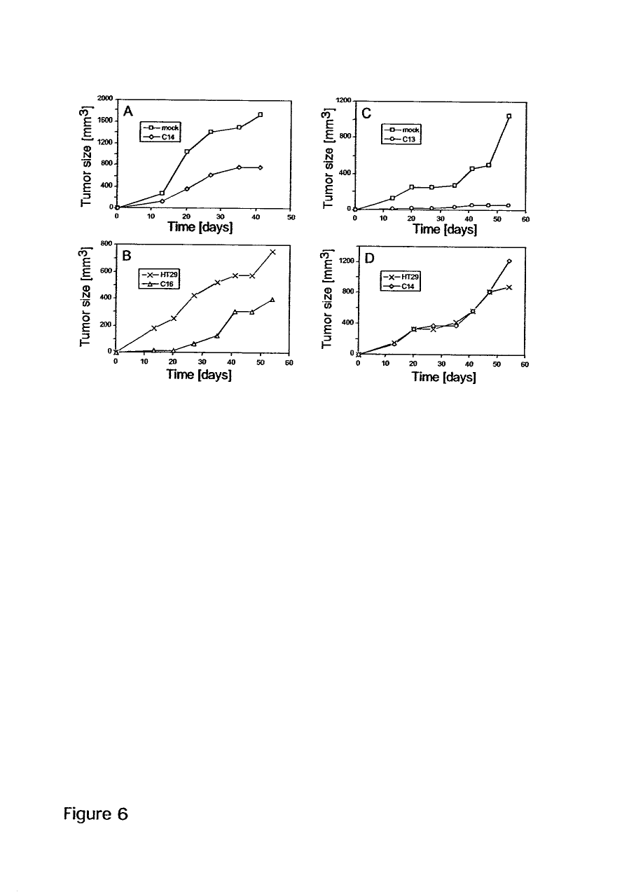

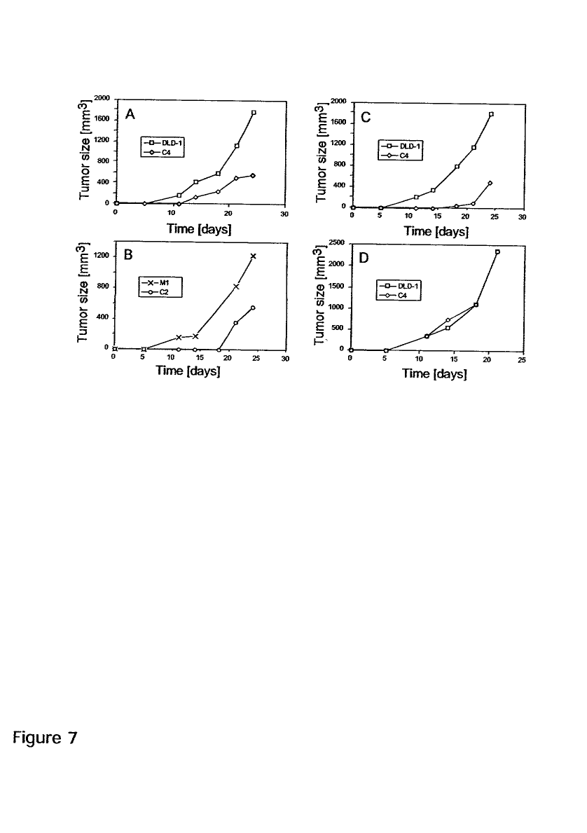

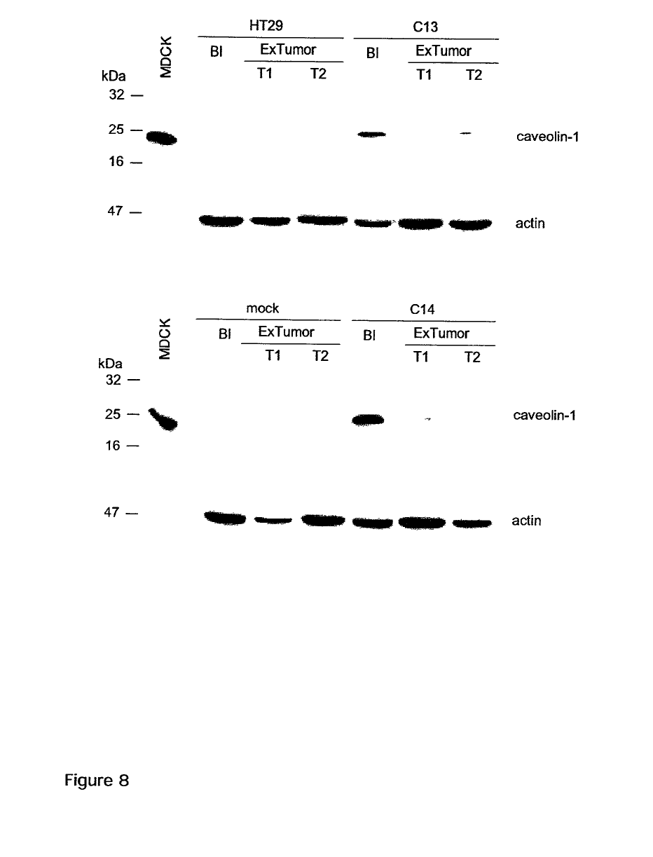

Finally, direct evidence for the importance of caveolin-1 in limiting the tumor forming ability of colon carcinoma cells is provided in FIGS. 6 and 7. Expression of caveolin-1 in transfected HT29 and DLD1 clones generally reduced the size of tumors formed upon injection into nude mice and delayed onset of tumor formation in most cases (FIG. 6 and 7). When tumors were detectable, their presence correlated with a decrease in basal caveolin-1 expression with respect to levels detected before injection into mice (FIG. 8). These observations provide strong support for the notion that an initial period of selection exists. Those cells that have lower caveolin-1 levels and/or succeed in reducing caveolin-1 expression subsequently proliferate and are able to form tumors in nude mice.

The reduction of caveolin-1 mRNA levels observed in breast and lung tumor cell lines indicates that caveolin-1 downregulation occurs primarily at the transcriptional level. The caveolin-1 gene is likely not methylated, in either breast primary tumors or tumor derived cell lines, indicating that the observed downregulation of caveolin-1 mRNA expression in breast tumors does not result from transcriptional silencing or by DNA methylation during tumor progression (20). A CpG island has been identified within the caveolin-1 promoter region that was methylated in human breast cancer cell lines (37). Similarly, hypermethylation of the caveolin-1 gene promoter has shown in prostate cancer. (See Prostate. 2001 Feb 15;46(3):249-56). Reduction of mRNA levels appeared to be an important mechanism by which caveolin-1 protein levels were regulated, since both were dramatically reduced in colon carcinoma cell lines as compared to levels observed in normal colon tissue (FIGS. 1 and 2).

In summary, the following have been observed: I) caveolin-1 protein levels were reduced in colon tumors from human patients; 2) colon carcinoma cells had low levels of caveolin-1 mRNA and protein; 3) expression of caveolin-1 in the colon carcinoma lines HT-29 and DLD1 blocked or retarded tumor formation in nude mice; 4) the ability of HT29-cav-1, DLD1-cav-1 (and also NIH-3T3 cells) to form tumors in nude mice, despite initial caveolin-1 presence, was linked to a selection process favoring proliferation of those cells with reduced basal caveolin-1 levels; 5) initial caveolin-1 down-regulation in colon carcinoma cells need not be an entirely irreversible event, since cell survival upon selection for either drug resistance or increased metastatic potential may require re-expression of caveolin-l.

Caveolin Nucleic Acids and Polypeptides

The present invention provides a nucleic acid molecule encoding the caveolin protein of the invention. As used herein, the terms polypeptide and protein are interchangeable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologues thereof. The nucleic acid molecule may be single-stranded or double-stranded, but preferably is comprised double-stranded DNA.

A caveolin-1 nucleic acid can encode a mature caveolin-1 polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to caveolin-1 nucleotide sequences can be prepared by standard synthetic techniques, e.g., using an automated DNA synthesizer.

In addition to naturally-occurring allelic variants of caveolin-1 sequences that may exist in the population, the skilled artisan will further appreciate that changes can be introduced by mutation into the nucleotide sequences of caveolin-i thereby leading to changes in the amino acid sequences of the encoded caveolin-1 proteins, without altering the functional ability of said caveolin-1 proteins.

Antisense nucleic acids

Another aspect of the invention pertains to isolated antisense nucleic acid molecules that are hybridizable to or complementary to the nucleic acid molecule comprising the nucleotide sequence of caveolin-1, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, 3 0 beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Vectors

Another aspect of the invention pertains to vectors, preferably expression vectors, containing a nucleic acid encoding a caveolin-1 protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Fusion Proteins

The invention also provides caveolin-1 chimeric or fusion proteins. As used herein, a caveolin-1 “chimeric protein” or “fusion protein” comprises a caveolin-1 polypeptide operatively-linked to a non-caveolin-1 polypeptide. An “caveolin-1 polypeptide” refers to a polypeptide having an amino acid sequence corresponding to a caveolin-1 protein, whereas a “non-caveolin-1 polypeptide” refers to a polypeptide having an amino acid sequence corresponding to a protein that is not substantially homologous to the caveolin-1 protein, e.g., a protein that is different from the caveolin-1 protein and that is derived from the same or a different organism.

Antibodies

Also included in the invention are antibodies to caveolin-1 proteins, or fragments of caveolin-1 proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Pharmaceutical Compositions

The caveolin-1 nucleic acid molecules, caveolin-1 proteins, and anti-caveolin-1 antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a caveolin-1 protein or anti-caveolin-1 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,81 1.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl.. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening Assays

The invention provides a method (also referred to herein as a “screening assay”) for identifying modulators, i.e., candidate or test compounds or agents (e.g., peptides, peptidomimetics, small molecules or other drugs) that bind to caveolin-1 proteins or have a stimulatory or inhibitory effect on, e.g., caveolin-1 protein expression or caveolin-1 protein activity. The invention also includes compounds identified in the screening assays described herein.

In one embodiment, the invention provides assays for screening candidate or test compounds, which bind to or modulate the activity of the membrane-bound form of a caveolin-1 protein or polypeptide or biologically-active portion thereof such as the scaffold domain. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J Mol. Biol. 222: 301-310; Ladner, U.S. Pat. No. 5,233,409.).

In one embodiment, an assay is a cell-based assay in which a cell which expresses a membrane-bound form of caveolin-1 protein, or a biologically-active portion thereof such as the scaffold domain, on the cell surface is contacted with a test compound and the ability of the test compound to bind to a caveolin-1 protein determined. The cell, for example, can be of mammalian origin or a yeast cell. Determining the ability of the test compound to bind to the caveolin-1 protein can be accomplished, for example, by coupling the test compound with a radioisotope or enzymatic label such that binding of the test compound to the caveolin-1 protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of caveolin-1 protein, or a biologically-active portion thereof, on the cell surface with a known compound which binds caveolin-1 to form an assay mixture, contacting the assay mixture with a test compound, and determining the ability of the test compound to interact with a caveolin-1 protein, wherein determining the ability of the test compound to interact with a caveolin-1 protein comprises determining the ability of the test compound to preferentially bind to caveolin-1 protein or a biologically-active portion thereof as compared to the known compound.

In another embodiment, an assay is a cell-based assay comprising contacting a cell expressing a membrane-bound form of caveolin-1 protein, or a biologically-active portion thereof such as the scaffold domain, on the cell surface with a test compound and determining the ability of the test compound to modulate (e.g., stimulate or inhibit) the activity of the caveolin-1 protein or biologically-active portion thereof. Determining the ability of the test compound to modulate the activity of caveolin-1 or a biologically-active portion thereof can be accomplished, for example, by determining the ability of the caveolin-1 protein to bind to or interact with a caveolin-1 target molecule. As used herein, a “target molecule” is a molecule with which a caveolin-1 protein binds or interacts in nature, for example, a molecule on the surface of a cell which expresses a caveolin-1 interacting protein, a molecule on the surface of a second cell, a molecule in the extracellular milieu, a molecule associated with the internal surface of a cell membrane or a cytoplasmic molecule. a caveolin-1 target molecule can be a non-caveolin-1 molecule or a caveolin-1 protein or polypeptide of the invention. In one embodiment, a caveolin-1 target molecule is a component of a signal transduction pathway that facilitates transduction of an extracellular signal (e.g. a signal generated by binding of a compound to a membrane-bound caveolin-1 molecule) through the cell membrane and into the cell. The target, for example, can be a second intercellular protein that has catalytic activity or a protein that facilitates the association of downstream signaling molecules with caveolin-1.

Determining the ability of the caveolin-1 protein to bind to or interact with a caveolin-1 target molecule can be accomplished by one of the methods described above for determining direct binding. In one embodiment, determining the ability of the caveolin-1 protein to bind to or interact with a caveolin-1 target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a caveolin-1 -responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a caveolin-1 protein or biologically-active portion thereof with a test compound and determining the ability of the test compound to bind to the caveolin-1 protein or biologically-active portion thereof. Binding of the test compound to the caveolin-1 protein can be determined either directly or indirectly as described above. In one such embodiment, the assay comprises contacting the caveolin-1 protein or biologically-active portion thereof with a known compound which binds caveolin-1 to form an assay mixture, contacting the assay mixture with a test compound, and determining the ability of the test compound to interact with a caveolin-1 protein, wherein determining the ability of the test compound to interact with a caveolin-1 protein comprises determining the ability of the test compound to preferentially bind to caveolin-1 or biologically-active portion thereof as compared to the known compound.

In still another embodiment, an assay is a cell-free assay comprising contacting caveolin-1 protein or biologically-active portion thereof such as the scaffold domain with a test compound and determining the ability of the test compound to modulate (e.g. stimulate or inhibit) the activity of the caveolin-1 protein or biologically-active portion thereof. Determining the ability of the test compound to modulate the activity of caveolin-1 can be accomplished, for example, by determining the ability of the caveolin-1 protein to bind to a caveolin-1 target molecule by one of the methods described above for determining direct binding. In an alternative embodiment, determining the ability of the test compound to modulate the activity of caveolin-1 protein can be accomplished by determining the ability of the caveolin-1 protein further modulate a caveolin-1 target molecule. For example, the catalytic/enzymatic activity of the target molecule on an appropriate substrate can be determined as described, supra.

In yet another embodiment, the cell-free assay comprises contacting the caveolin-1 protein or biologically-active portion thereof with a known compound which binds caveolin-1 protein to form an assay mixture, contacting the assay mixture with a test compound, and determining the ability of the test compound to interact with a caveolin-1 protein, wherein determining the ability of the test compound to interact with a caveolin-1 protein comprises determining the ability of the caveolin-1 protein to preferentially bind to or modulate the activity of a caveolin-1 target molecule.

The cell-free assays of the invention are amenable to use of both the soluble form and the membrane-bound form of caveolin-1 protein. In the case of cell-free assays comprising the membrane-bound form of caveolin-1 protein, it may be desirable to utilize a solubilizing agent such that the membrane-bound form of caveolin-1 protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X- 114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either caveolin-1 protein or its target molecule to facilitate separation of complexed from uncomplexed forms of one or both of the proteins, as well as to accommodate automation of the assay. Binding of a test compound to caveolin-1 protein, or interaction of caveolin-1 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-caveolin-1 fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or caveolin-1 protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of caveolin-1 protein binding or activity determined using standard techniques.

Other techniques for immobilizing proteins on matrices can also be used in the screening assays of the invention. For example, either the caveolin-1 protein or its target molecule can be immobilized utilizing conjugation of biotin and streptavidin. Biotinylated caveolin-1 protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with caveolin-1 protein or target molecules, but which do not interfere with binding of the caveolin- I protein to its target molecule, can be derivatized to the wells of the plate, and unbound target or caveolin-1 protein trapped in the wells by antibody conjugation. Methods for detecting such complexes, in addition to those described above for the GST-immobilized complexes, include immunodetection of complexes using antibodies reactive with the caveolin-1 protein or target molecule, as well as enzyme-linked assays that rely on detecting an enzymatic activity associated with the caveolin-1 protein or target molecule.

In another embodiment, modulators of caveolin-1 protein expression are identified in a method wherein a cell is contacted with a candidate compound and the expression of caveolin-1 mRNA or protein in the cell is determined. The level of expression of caveolin-1 mRNA or protein in the presence of the candidate compound is compared to the level of expression of caveolin-1 mRNA or protein in the absence of the candidate compound. The candidate compound can then be identified as a modulator of caveolin-1 mRNA or protein expression based upon this comparison. For example, when expression of caveolin-1 mRNA or protein is greater (i.e., statistically significantly greater) in the presence of the candidate compound than in its absence, the candidate compound is identified as a stimulator of caveolin-1 mRNA or protein expression. Alternatively, when expression of caveolin-1 mRNA or protein is less (statistically significantly less) in the presence of the candidate compound than in its absence, the candidate compound is identified as an inhibitor of caveolin-1 mRNA or protein expression. The level of caveolin-1 mRNA or protein expression in the cells can be determined by methods described herein for detecting caveolin-1 mRNA or protein.

In yet another aspect of the invention, the caveolin-1 proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with caveolin-1 (“caveolin-1-binding proteins” or “caveolin-1-bp”) and modulate caveolin-1 activity. Such caveolin-1-binding proteins are also likely to be involved in the propagation of signals by the caveolin-1 proteins as, for example, upstream or downstream elements of the caveolin- l pathway.

The two-hybrid system is based on the modular nature of most transcription factors, which consist of separable DNA-binding and activation domains. Briefly, the assay utilizes two different DNA constructs. In one construct, the gene that codes for caveolin-1 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a caveolin-1-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein, which interacts with caveolin-1.

The invention further pertains to novel agents identified by the aforementioned screening assays and uses thereof for treatments as described herein.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining caveolin-1 protein and/or nucleic acid expression as well as caveolin-1 activity, in the context of a biological sample (e.g., blood, serum, cells, tissue) to thereby determine whether an individual is afflicted with a disease or disorder, or is at risk of developing a disorder, associated with aberrant caveolin-1 expression or activity. The disorders include cell proliferative disorders such as cancer. The invention also provides for prognostic (or predictive) assays for determining whether an individual is at risk of developing a disorder associated with caveolin-1 protein, nucleic acid expression or activity. For example, mutations in a caveolin-1 gene can be assayed in a biological sample. Such assays can be used for prognostic or predictive purpose to thereby prophylactically treat an individual prior to the onset of a disorder characterized by or associated with caveolin-1 protein, nucleic acid expression, or biological activity.

Another aspect of the invention provides methods for determining caveolin-1 protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of caveolin-1 in clinical trials.

Diagnostic Assays

An exemplary method for detecting the presence or absence of caveolin- 1 in a biological sample involves obtaining a biological sample from a test subject and contacting the biological sample with a compound or an agent capable of detecting caveolin-1 protein or nucleic acid (e.g., mRNA, genomic DNA) that encodes caveolin-1 protein such that the presence of caveolin-1 is detected in the biological sample. An agent for detecting caveolin-1 mRNA or genomic DNA is a labeled nucleic acid probe capable of hybridizing to caveolin-1 mRNA or genomic DNA. The nucleic acid probe can be, for example, a full-length caveolin-1 nucleic acid, such as the nucleic acid of caveolin-1, or a portion thereof, such as an oligonucleotide of at least 15, 30, 50, 100, 250 or 500 nucleotides in length and sufficient to specifically hybridize under stringent conditions to caveolin-1 mRNA or genomic DNA. Other suitable probes for use in the diagnostic assays of the invention are described herein.

An agent for detecting caveolin-1 protein is an antibody capable of binding to caveolin-1 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect caveolin-1 mRNA, protein, or genomic DNA in a biological sample in vitro as well as in vivo. For example, in vitro techniques for detection of caveolin-1 mRNA include Northern hybridization's and in situ hybridization's. In vitro techniques for detection of caveolin-1 protein include enzyme linked immunosorbent as (ELISA), Western blot, immunoprecipitation, and immunofluorescence. In vitro techniques for detection of caveolin-1 genomic DNA include Southern hybridization. Furthermore, in vivo techniques for detection of caveolin-1 protein include introducing into a subject a labeled anti-caveolin-1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting caveolin-1 protein, mRNA, or genomic DNA, such that the presence of caveolin-1 protein, mRNA or genomic DNA is detected in the biological sample, and comparing the presence of caveolin-1 protein, mRNA or genomic DNA in the control sample with the presence of caveolin-1 protein, mRNA or genomic DNA in the test sample.

The invention also encompasses kits for detecting the presence of caveolin-1 in a biological sample. For example, the kit can comprise: a labeled compound or agent capable of detecting caveolin-1 protein or mRNA in a biological sample; means for determining the amount of caveolin-1 in the sample; and means for comparing the amount of caveolin-1 in the sample with a standard. The compound or agent can be packaged in a suitable container. The kit can further comprise instructions for using the kit to detect caveolin-1 protein or nucleic acid.

Prognostic Assays

The diagnostic methods described herein can furthermore be utilized to identify subjects having or at risk of developing a disease or disorder associated with aberrant caveolin-1 expression or activity. For example, the assays described herein, such as the preceding diagnostic assays or the following assays, can be utilized to identify a subject having or at risk of developing a disorder associated with caveolin-1 protein, nucleic acid expression or activity. Alternatively, the prognostic assays can be utilized to identify a subject having or at risk for developing a disease or disorder. Thus, the invention provides a method for identifying a disease or disorder associated with aberrant caveolin-1 expression or activity in which a test sample is obtained from a subject and caveolin-1 protein or nucleic acid (e.g., mRNA, genomic DNA) is detected, wherein the presence of caveolin-i protein or nucleic acid is diagnostic for a subject having or at risk of developing a disease or disorder associated with aberrant caveolin-1 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant caveolin-1 expression or activity. For example, such methods can be used to determine whether a subject can be effectively treated with an agent for a disorder. Thus, the invention provides methods for determining whether a subject can be effectively treated with an agent for a disorder associated with aberrant caveolin-1 expression or activity in which a test sample is obtained and caveolin-1 protein or nucleic acid is detected (e g., wherein the presence of caveolin-1 protein or nucleic acid is diagnostic for a subject that can be administered the agent to treat a disorder associated with aberrant caveolin-1 expression or activity).

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a caveolin-1 gene.

Furthermore, any cell type or tissue, preferably peripheral blood leukocytes, in which caveolin-1 is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on caveolin-1 activity (e.g., caveolin-1 gene expression), as identified by a screening assay described herein can be administered to individuals to treat (prophylactically or therapeutically) disorders (The disorders include cell proliferative disorders such as cancer.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of caveolin-1 protein, expression of caveolin-1 nucleic acid, or mutation content of caveolin-1 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of caveolin-1 protein, expression of caveolin-1 nucleic acid, or mutation content of caveolin-1 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a caveolin-1 modulator, such as a modulator identified by one of the exemplary screening assays described herein.

Monitoring of Effects During Clinical Trials

Monitoring the influence of agents (e.g., drugs, compounds) on the expression or activity of caveolin-1 (e.g., the ability to modulate aberrant cell proliferation and/or differentiation) can be applied not only in basic drug screening, but also in clinical trials. For example, the effectiveness of an agent determined by a screening assay as described herein to increase caveolin-1 gene expression, protein levels, or upregulate caveolin-1 activity, can be monitored in clinical trails of subjects exhibiting decreased caveolin-1 gene expression, protein levels, or downregulated caveolin-1 activity. Alternatively, the effectiveness of an agent determined by a screening assay to decrease caveolin-1 gene expression, protein levels, or downregulate caveolin-1 activity, can be monitored in clinical trails of subjects exhibiting increased caveolin-I gene expression, protein levels, or upregulated caveolin-1 activity. In such clinical trials, the expression or activity of caveolin-1 and, preferably, other genes that have been implicated in, for example, a cellular proliferation or immune disorder can be used as a “read out” or markers of the immune responsiveness of a particular cell.

By way of example, and not of limitation, genes, including caveolin-1, that are modulated in cells by treatment with an agent (e.g., compound, drug or small molecule) that modulates caveolin-1 activity (e.g., identified in a screening assay as described herein) can be identified. Thus, to study the effect of agents on cellular proliferation disorders, for example, in a clinical trial, cells can be isolated and RNA prepared and analyzed for the levels of expression of caveolin-1 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of caveolin-1 or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule. or other drug candidate identified by the screening assays described herein) comprising the steps of (i) obtaining a pre-administration sample from a subject prior to administration of the agent; (ii) detecting the level of expression of a caveolin-1 protein, mRNA, or genomic DNA in the preadministration sample; (iii) obtaining one or more post-administration samples from the subject; (iv) detecting the level of expression or activity of the caveolin-1 protein, mRNA, or genomic DNA in the post-administration samples; (v) comparing the level of expression or activity of the caveolin-1 protein, mRNA, or genomic DNA in the pre-administration sample with the caveolin-1 protein, mRNA, or genomic DNA in the post administration sample or samples; and (vi) altering the administration of the agent to the subject accordingly. For example, increased administration of the agent may be desirable to increase the expression or activity of caveolin-1 to higher levels than detected, i.e., to increase the effectiveness of the agent. Alternatively, decreased administration of the agent may be desirable to decrease expression or activity of caveolin-1 to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant caveolin-1 expression or activity. The disorders include, but are not limited to cell proliferative disorders such as cancer.

Disease and Disorders

Diseases and disorders that are characterized by increased (relative to a subject not suffering from the disease or disorder) levels or biological activity may be treated with therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant caveolin-1 expression or activity, by administering to the subject an agent that modulates caveolin-1 expression or at least one caveolin-1 activity. Subjects at risk for a disease that is caused or contributed to by aberrant caveolin-1 expression or activity can be identified by, for example, any or a combination of diagnostic or prognostic assays as described herein. Administration of a prophylactic agent can occur prior to the manifestation of symptoms characteristic of the caveolin-1 aberrancy, such that a disease or disorder is prevented or, alternatively, delayed in its progression. Depending upon the type of caveolin-1 aberrancy, for example, a caveolin-1 agonist or caveolin-1 antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating caveolin-1 expression or activity for therapeutic purposes. The modulatory method of the invention involves contacting a cell with an agent that modulates one or more of the activities of caveolin-1 protein activity associated with the cell. An agent that modulates caveolin-1 protein activity can be an agent as described herein, such as a nucleic acid or a protein, a naturally-occurring cognate ligand of a caveolin-1 protein, a peptide, a caveolin-1 peptidomimetic, or other small molecule. In one embodiment, the agent stimulates one or more caveolin-1 protein activity. Examples of such stimulatory agents include active caveolin-1 protein and a nucleic acid molecule encoding caveolin-1 that has been introduced into the cell. In another embodiment, the agent inhibits one or more caveolin-1 protein activity. Examples of such inhibitory agents include antisense caveolin-1 nucleic acid molecules and anti-caveolin-1 antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a caveolin-1 protein or nucleic acid molecule. In one embodiment, the method involves administering an agent (e.g., an agent identified by a screening assay described herein), or combination of agents that modulates (e.g., up-regulates or down-regulates) caveolin-1 expression or activity. In another embodiment, the method involves administering a caveolin-1 protein or nucleic acid molecule as therapy to compensate for reduced or aberrant caveolin-1 expression or activity.

Stimulation of caveolin-1 activity is desirable in situations in which caveolin-1 is abnormally downregulated and/or in which increased caveolin-1 activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The caveolin-1 nucleic acids and proteins of the invention are useful in potential prophylactic and therapeutic applications implicated in a variety of disorders including, but not limited to cell proliferative disorders such as cancer.

As an example, a cDNA encoding the caveolin-1 protein of the invention may be useful in gene therapy, and the protein may be useful when administered to a subject in need thereof. By way of non-limiting example, the compositions of the invention will have efficacy for treatment of patients suffering from: cell proliferative disorders such as cancer.

Both the novel nucleic acid encoding the caveolin-1 protein, and the caveolin-1 protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

The following experiments are offered to illustrate embodiments of the invention and should not be viewed as limiting the scope of the invention.

### Example 1: Materials and Methods

Reagents and antibodies. Dulbecco's modified Eagle's medium (DMEM), RPMI-1640, trypsin/EDTA, antibiotics (PSN: penicillin, streptomycin, neomycin) were purchased from Life Technologies (Paisley, Scotland). Fetal calf serum (FCS) was from Seromed-Biochrom KG (Berlin, Germany), isopropyl β-D-thiogalactoside (IPTG) from Eurogentec (Seraing, Belgium), hygromycin B from Calbiochem (La Jolla, USA). The BCA protein determination kit was from Pierce (Rockford, USA), prestained molecular weight protein markers were from New England Biolab Inc. (Beverly, USA), and the enhanced chemiluminesence (ECL) kit was from Amersham International (Bucks, UK). The polyclonal anti-caveolin-1 antibody (C13630) was purchased from Transduction Laboratories (Lexington, USA) and the monoclonal anti-actin antibody (010056) was from Bioscience (Seikagu Corporation, Tokyo, Japan). Goat anti-rabbit (1706515) and Goat anti-mouse (A4416) antibodies coupled to horseradish-peroxidase (HRP) were from Bio-Rad Laboratories (Hercules, USA) and Sigma (St-Louis, USA), respectively.

Cell culture. The human colon carcinoma cell lines SW480, SW620, Co112, HT29 and its derived differentiated clones HT29-5M12, HT29-5M21 (Lesuffleur, T., Barbat, A., Dussaulx, E., and Zweibaum, A. Growth adaptation to methotrexate of HT-29 human colon carcinoma cells is associated with their ability to differentiate into columnar absorptive and mucus-secreting cells, Cancer Res. 50: 6334-43, 1990), Lovo as well as the Lovo clones E2 and C5, selected for higher metastatic potential (Glenney, J. R., Jr. The sequence of human caveolin reveals identity with VIP21, a component of transport vesicles, FEBS Lett. 314. 45-8, 1992), were provided by Dr. Bernard Sordat (Swiss Institute for Cancer Research (ISREC), Epalinges), the line DLD1, by Dr. Emanuela Felley-Bosco (Inst. of Pharmacology, University of Lausanne) and the lines Caco2 and MDCK strain II, by Dr. Walter Hunziker (Inst. of Biochemistry, University of Lausanne). NIH-3T3 fibroblasts and NIH-3T3 ExTu, a population of NIH-3T3 cells which were isolated after tumor formation on nude mice (Peli, J., Schroter, M., Rudaz, C., Hahne, M., Meyer, C., Reichmann, E., and Tschopp, J. Oncogenic Ras inhibits Fas ligand-mediated apoptosis by downregulating the expression of Fas, EMBO J. 18: 1824-31, 1999) were provided by Dr Ernst Reichmann (ISREC, Epalinges). HT29, HT29-5M12, HT29-5M21, Col 12, Caco2, MDCK, NIH-3T3 and NIH-ExTu were cultured in DMEM supplemented with 10 % FCS and PSN. Lovo and Lovo clones were cultured in the same medium containing 0.1% Na₂CO₃. SW480, SW620 and DLD1 cells were maintained in RPMI-1640 with 10% FCS and antibiotics as above. All cells were cultured at 37° C. under 5% CO₂, and passaged every week using trypsin/EDTA.

Isolation of human colon crypts and purification of epithelial cells. Human colonic crypts, and subsequently colonic epithelial cells or stroma were isolated as described previously (Reymond, M. A., Sanchez, J. C., Schneider, C., Rohwer, P., Tortola, S., Hohenberger, W., Kirchner, T., Hochstrasser, D. F., and Kockerling, F. Specific sample preparation in colorectal cancer, Electrophoresis. 18: 622-4, 1997; Reymond, M. A., Sanchez, J. C., Hughes, G. J., Gunter, K., Riese, J., Tortola, S., Peinado, M. A., Kirchner, T., Hohenberger, W., Hochstrasser, D. F., and Kockerling, F. Standardized characterization of gene expression in human colorectal epithelium by two-dimensional electrophoresis, Electrophoresis. 18: 2842-8, 1997) after obtaining the informed consent of the patients. Operations were performed at the University Hospital of Geneva (Switzerland) and at the Carl-Thiem Klinikum of Cottbus (Germany). Authorization was provided by the Ethics Committee. Epithelial cell viability after purification was over 90% as determined by Trypan Blue staining. After cross-staining with a pan-anti-cytokeratin antibody (CAM 5,12, Perkin Elmer, Norwalls, USA) epithelial cell preparations were shown to be over 95% pure by FACS analysis.

Plasmids. Plasmid plac[OP-cav-l that allows IPTG inducible expression of caveolin-1 in transfected cells was constructed as follows. The full-length cDNA encoding dog caveolin-1 was amplified by RT-PCR using caveolin-1 specific primers flanked by Not I restriction sites and RNA isolated from MDCK cells as a template. Resulting cDNA was purified and then cloned into the Not I site of placIOP, which consists of vectors p3'SS and pOPRSVI CAT from Invitrogen (Carlsbad, USA) fused together as described (Peli, J., Schroter, M., Rudaz, C., Hahne, M., Meyer, C., Reichmann, E., and Tschopp, J. Oncogenic Ras inhibits Fas ligand-mediated apoptosis by downregulating the expression of Fas, EMBO J. 18: 1824-31, 1999). The sequence of the 5′ sense primer, which in addition included a Kozak motif (underlined) upstream of the initiation ATG codon, was 5′-CCGAGCGCGGCCGCCATGTCTGGGGCAAATAC-3′ (SEQ ID NO: 1) and that of the anti-sense primer was 5′-TATCTGGCGGCCGCTTATGTTTCTTTC-TGCATGTTG-3′ (SEQ ID NO: 2). Not I restriction sites are indicated in bold. The construct pGEM-cav-1 was used to produced caveolin-1 specific probes for Northern analysis and obtained by amplifying a cDNA sequence conserved between dog and human (nucleotides 63-433 of the eDNA coding sequence) by RT-PCR using RNA isolated from MDCK cells as a template and appropriate primers to allow subsequent cloning of the amplified product into the Xba I/Eco RI sites of pGEM 2 (Promega, Madison, USA). The sense primer included a Xba I site (bold): 5′-GGGCAACATCTAGAAGCCCAACAAC-3′ (SEQ ID NO: 3). The anti-sense primer contained an EcoRI site (bold): 5′-CTGATGCACTGAATTCCAATCAGGAA-3′ (SEQ ID NO: 4). The pSP65m-β-actin plasmid (Plaetinck, G., Combe, M. C., Corthesy, P., Sperisen, P., Kanamori, H., Honjo, T., and Nabholz, M. Control of IL-2 receptor-alpha expression by IL-1, tumor necrosis factor, and IL-2. Complex regulation via elements in the 5′ flanking region, J. Immunol. 145: 3340-7, 1990. 29) used for standardization of Northern blots was kindly provided by Markus Nabholz (ISREC, Epalinges).

Stable transfection of HT29 and DLD1 cells with a plasmid permitting inducible expression of caveolin-1. HT29 and DLD1 cells were stably transfected with placlOP (mock) or placlOP-cav-1 by calcium phosphate precipitation as described (Hunziker, W. and Mellman, I. Expression of macrophage-lymphocyte Fc receptors in Madin-Darby canine kidney cells: polarity and transcytosis differ for isoforms with or without coated pit localization domains, J. Cell Biol. 109: 3291-302, 1989). Individual clones resistant to 500 μg/ml hygromycin B were screened for IPTG induced expression of recombinant caveolin-1 by Western blot analysis. Induction of caveolin-1 was maximal after 24 h of stimulation with 1 mM IPTG.

Northern analysis. Total cellular RNA was extracted with a purification kit in the presence of guanidinium thiocyanate (Qiagen, Hilden, Germany) according to the manufacturer's instructions. Samples containing 15 μg of cytoplasmic RNA were fractionated on 1% agarose gels prepared in 10 mM sodium phosphate buffer pH 7, transferred on a nylon membrane and cross-linked to the membrane by UV irradiation as described (Sambrook, J. F., Fritsch, E. F., and Maniatis, T. Molecular cloning: a laboratory manual. New York: Cold Spring Harbor Laboratory Press, 1989; Melton, D. A., Krieg, P. A., Rebagliati, M. R., Maniatis, T., Zinn, K., and Green, M. R. Efficient in vitro synthesis of biologically active RNA and RNA hybridization probes from plasmids containing a bacteriophage SP6 promoter, Nucleic Acids Res. 12: 7035-56, 1984). Alternatively, multiple tissue or cell line northern blots were purchased from Clontech Laboratories (Palo Alto, USA). After over-night pre-incubation at 55° C. in hybridization buffer (50% formamide, 5×SSC, 1 mM EDTA, 0.2% SDS, 2×Denhardt's, 0.5 mg/ml yeast tRNA, 0.25 mg/ml salmon sperm DNA in 50 mM sodium phosphate buffer pH 6.5), blots were further incubated 24 h with 10⁶ cpm/ml [³²P]labeled RNA probes for caveolin-1 in hybridization buffer. Probes were synthesized as described (Sambrook, J. F., Fritsch, E. F., and Maniatis, T. Molecular cloning: a laboratory manual. New York: Cold Spring Harbor Laboratory Press, 1989; Melton, D. A., Krieg, P. A., Rebagliati, M. R., Maniatis, T., Zinn, K., and Green, M. R. Efficient in vitro synthesis of biologically active RNA and RNA hybridization probes from plasmids containing a bacteriophage SP6 promoter, Nucleic Acids Res. 12. 7035-56, 1984) from Xba I linearized pGEM-cav-1. Blots were washed four times 15 min at 65° C. in 0.1×SSC, 0.1% SDS solution and exposed to film (BioMax MR-1, Kodak, New York, USA). After caveolin-1 detection, blots were stripped (according to a protocol from Clontech Laboratories, Palo Alto, USA) and standardized to β-actin using a ribo-probe prepared from Mae I linearized pSP65m-β-actin.

SDS-PAGE and Western blotting. Expression of caveolin-1 in carcinoma cell lines, transfected HT29 or DLD1 cells, human colon tissues or NIH-3T3 cells was studied by Western blot analysis. Cells were grown until they were 80% confluent. Culture medium was then removed, the cells were washed twice with cold PBS and lyzed in buffer containing 4% SDS, 125 mM Tris-HCl pH 6.8 and protease inhibitors (10 μg/ml benzamidine, 2 μg/ml antipain, 1 μg/ml leupeptin). Cell lysates were sonicated and the protein concentration determined with the BCA assay. Human colon tissues were lyzed similarly but homogenates were passed several times through a 25-G needle, sonicated and cleared by centrifugation for 5 min at 10'000×g in an Eppendorf centrifuge. The protein concentration of supernatants was determined by the BCA assay. All samples were adjusted to Laemmli buffer composition (Laemmli, U. K. Cleavage of structural proteins during the assembly of the head of bacteriophage T4, Nature. 227. 680-5, 1970) (2 % SDS, 10 % glycerol, 62.5 mM Tris-HCl pH 6.8, 100 mM DTT and 0.1% bromophenol blue), denatured by heating at 95° C. for 5 min and subsequently loaded on 10% gels. After separation, proteins were transferred onto nitrocellulose. Membranes were stained with Ponceau Red S (Sigma, St-Louis, USA) to verify equal loading of samples and blocked overnight in PBS/3% milk/2 mM NaN3. Then membranes were incubated 1 h at RT with either anti-caveolin-1 (1:10'000) or anti-actin (1:1'000) antibodies diluted in blocking solution. Membranes were then washed 5 times in PBS/0.1% Tween-20, incubated 1 h with second antibody (1:2500) diluted in blocking solution (no azide) and washed again as before. Membrane-bound second antibodies were detected by ECL following instructions of the manufacturer.

Tumorigenicity assays. 10⁶ cells were suspended in 50 μl DMEM and injected subcutaneously into 6-8 week old nude mice. For each mouse, control cells (parental HT29 or DLD1 cells or mock transfected cells) were injected on the left and HT29 or DLD1 cells transfected with caveolin-1 (clones C13, C14, C16 or C2, C4 respectively) on the right. Large (D) and small (d) diameters of growing tumors were measured twice a week and corresponding volumes (V) were estimated using the equation V=d2×D×π/6. To re-isolate tumor cells for further culture, the tumor tissue was excised, cut into small pieces under sterile conditions using scalpel blades and digested with trypsin/EDTA for 15 min at 37° C. Tumor cells were cultured until confluent in 10 cm Petri dishes, trypsinized, diluted 1:10 in fresh medium and seeded again. After a second passage, when tissue debris and contaminating cells had been eliminated, ex-tumor cells were lyzed at 80% confluency and processed for caveolin-1 detection as described.

### Example 2: Caveolin-1 Expression in Normal Human Colon Tissue and Colon Carcinomas

Caveolin-1 mRNA and protein levels were analyzed in a variety of human colon carcinoma cell lines and in human tissues of normal or tumor origin. In initial experiments, caveolin-1 mRNA levels in human tissues (epithelium of the small intestine and colon) and the SW480 carcinoma cell line (FIG. 1, upper panel), were compared by Northern blotting analysis. The 3 kb specific mRNA of caveolin-1 (Glenney, J. R., Jr. The sequence of human caveolin reveals identity with VIP21, a component of transport vesicles, FEBS Lett. 314. 45-8, 1992) was extremely abundant in heart, but undetectable in peripheral blood leukocytes (PBL) which served in these experiments as positive and negative controls, respectively (Glenney, J. R., Jr. The sequence of human caveolin reveals identity with VIP21, a component of transport vesicles, FEBS Lett. 314: 45-8, 1992; Fra, A. M., Williamson, E., Simons, K., and Parton, R. G. Detergent-insoluble glycolipid microdomains in lymphocytes in the absence of caveolae, J. Biol. Chem. 269: 30745-8, 1994). Interestingly, caveolin-1 mRNA levels in small intestine or in colon were 10-20 fold higher than the levels detected in the colon carcinoma cell line SW480.

To test whether this might represent a general characteristic of colon carcinoma lines, levels of caveolin-1 protein and mRNA were compared by Western blot analysis (FIG. 2). Caveolin-1 protein levels (FIG. 2A) were extremely low in all lines analyzed, as compared to MDCK, and never exceeded the levels observed in SW480. Expression was particularly low in HT29, Col 12 and Caco2. Even for SW480, SW620 and DLD1 where caveolin-T was present, levels were 50-100-fold lower than in MDCK cells. In addition, all colon carcinoma cell lines had low levels of caveolin-1 mRNA (FIG. 2B), comparable to or lower than those observed with SW480 (FIG. 1).

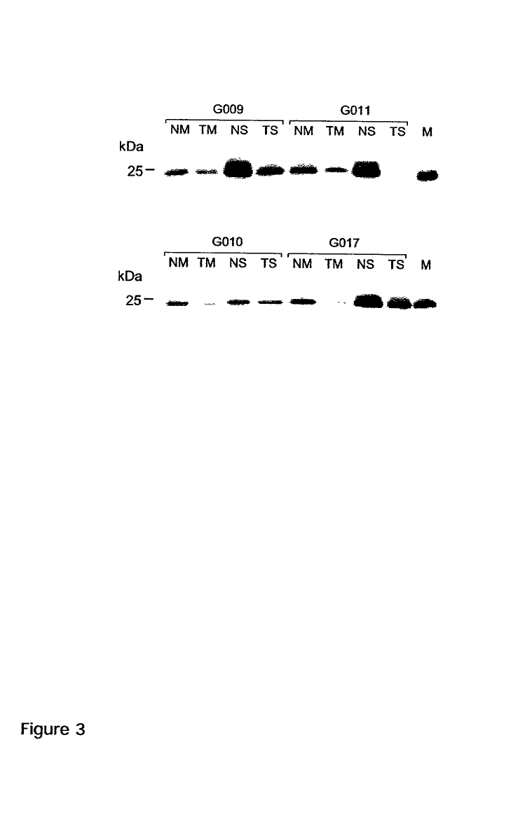

To underscore the importance of the above findings in colon carcinoma cell lines, both normal and tumor tissues samples from patients with colon carcinomas were examined. Results from four patients (codes G009, G010, G011 and G017) are shown (FIG. 3), whereby colon mucosa (epithelium) and stroma from either normal or tumor tissue were compared. Caveolin-1 levels were decreased in tumor mucosa, suggesting that low levels of caveolin-1 expression detected in colon carcinoma lines reflect an inherent property of the immortalized cells that were derived from colon tumor epithelium. In addition, caveolin-1 levels were similarly decreased in tumor stroma.

Several more patients (total n=15) were characterized in a similar fashion by Western blotting analysis. To facilitate the comparison, caveolin-1 signals obtained were quantified by scanning densitometry. Numbers shown for mucosa and stroma (Table 1) are equivalent to the ratio of scanning densitometry values obtained for normal versus tumor tissue. In about 70% (10/15) of the samples analyzed, caveolin-T levels were reduced up to 7-fold (average 3.9-fold) in mucosa. For stroma, fewer samples were available, but on an average a similar decrease in caveolin-1 presence was observed. In about 30% (5/15) of the cases, either no significant decrease of caveolin-1 expression was observed in tumor tissue or the trend was even reversed in one situation.

TABLE 1
Caveolin-1 expression levels in human colon tissue. Analysis of samples
from patients with colon cancer.
Caveolin-1 expression levels^{a}

| | Tumor | normal mucosa/ | normal stroma/ |
|---|---|---|---|
| Patients^{b} | characteristics^{c} | tumor mucosa | tumor stroma |
| G006 | T4 N0 M0 | 3.3 | n.a^{d} |
| G009 | T4 N1 M0 | 3.4 | 5.2 |
| G010 | T4 N0 M0 | 3.3 | 3.0 |
| G011 | T4 N0 M1 | 5.1 | 6.4 |
| G012 | T4 N1 M1 | 6.4 | 2.5 |
| G013 | T3 N0 M0 | 2.0 | 0.7 |
| G016 | T2 N0 M0 | 2.2 | n.a |
| G017 | T3 N0 M0 | 5.4 | 2.8 |
| G019 | T4 N0 M0 | 1.2 | n.a |
| G022 | T4 N2 M1 | 0.7 | n.a |
| C021 | T3 N1 M0 | 2.2 | 1.1 |
| C022 | T3 N0 M0 | 0.7 | 2.5 |
| C023 | T2 N0 M0 | 0.5 | 2.2 |
| C024 | T3 N0 M0 | 2.8 | 3.3 |
| C025 | T2 N0 M0 | 0.9 | 1.4 |

^{a}Samples excised by surgery from colon cancer patients were analyzed by Western blotting as in FIG. 3 and relative levels of caveolin-1 expression were determined by scanning densitometry. For each patient, results are presented as a ratio between the levels of caveolin-1 measured in normal colon tissues (mucosa or stroma) divided be those detected in their tumor counterparts.
^{b}Patient data were anonymized
^{c}Tumors were characterized by staging criteria (TNM system) describing local spread of the primary tumor (T), metastasis of regional lymph nodes (N) and distant metastasis (M) [Hermanek, P., Hutter, R.V.P., Sobin, L.H., Wagner, G., and Wittekind, C. TNM-Atlas. Verlag, Heidelberg, New York: Springer, 1998]
^{d}n.a, samples were not available.

Taken together these results suggest that caveolin-1 expression levels are reduced in human colon tumor samples as well as colon carcinoma cell lines, and that decreased presence of the caveolin-1 protein may be attributed to reduced mRNA levels.

### Example 3: Caveolin-1 Downregulation Occurs During Tumor Formation

It was not clear at this point whether tumor formation itself is sufficient to reduce caveolin-1 expression. Since levels were low in colon carcinoma lines, a different model system was required. NIH-3T3 fibroblast cells are ideal in this respect, since they express caveolin-1 and are able to induce tumor formation in nude mice after extended periods of time, on the order of 50-60 days (Peli, J., Schroter, M., Rudaz, C., Hahne, M., Meyer, C., Reichmann, E., and Tschopp, J. Oncogenic Ras inhibits Fas ligand-mediated apoptosis by downregulating the expression of Fas, EMBO J. 18: 1824-31, 1999). Comparison by Western and Northern blotting of parental NIH-3T3 cells with cells isolated after tumor formation in nude mice clearly revealed that the latter expressed lower levels of caveolin-1 protein (FIG. 4A) and mRNA (FIG. 4B). Thus, tumor formation in mice correlated either with reduction of caveolin-1 expression in NIH-3T3 cells or elimination of cells expressing caveolin-1.

### Example 4: Caveolin-1 Can Be Re-Expressed in the Colon Carcinoma Cell Lines HT29 and DLD1

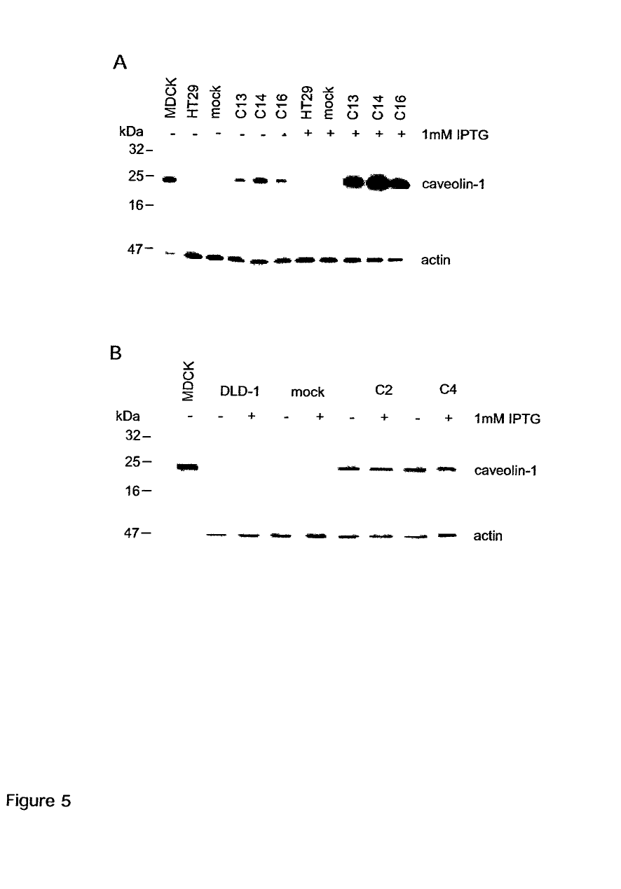

To assess whether the presence of caveolin-1 in colon carcinoma cells may represent a rate-limiting factor in tumor development of these cells, HT29 or DLD1 cells were transfected with a plasmid harboring a full-length dog caveolin-1 encoding cDNA under the control of an IPTG-inducible promoter (placIOP-cav-1). Several clones were isolated and checked for caveolin-1 expression by Western blotting (FIG. 5). The clones C13, C14 and C16 expressed higher caveolin-1 levels than parental HT29 or mock-transfected cells, even when grown in the absence of IPTG (FIG. 5A, IPTG). Addition of IPTG (FIG. 5A, +IPTG) dramatically increased the basal level of caveolin-1 expression in all clones but had no effect on both parental and mock-transfected HT29 cells. Basal caveolin-1 expression levels and levels after IPTG induction were not identical in these clones, with clone C14 expressing the highest and C16 the lowest amounts. Similarly, clones C2 and C4 expressed higher caveolin-1 levels than parental DLD1 and mock transfected cells, but caveolin-1 expression levels were not increased by the addition of IPTG (FIG. 5B).

### Example 5: Re-Expression of Caveolin-1 in HT29 and DLD1 Cells Reduced Tumor Formation in Nude Mice

The results obtained with NIH-3T3 cells in nude mice showed that caveolin-1 downregulation occurred upon tumor formation and suggested that re-introduction of caveolin-1 into colon carcinoma lines like HT29 or DLD1 may block the tumor forming ability of these cells. To test this hypothesis, nude mice were injected in each case with control cells (parental or mock transfected cells) on the left and HT29 (clones C13, C14, C16) or DLD1 (clones C2, C4) cells expressing caveolin-1 on the right side (total number of mice n=13 or n=7, respectively) (FIGS. 6 and 7). All HT29 cells led to noticeable tumor formation one month after injection, but in 75% (n=10) of the cases studied, tumors were either significantly smaller for caveolin-1 expressing HT29 clones (FIG. 6A, B; n=7) or almost undetectable (FIG. 6C; n=3). Where tumors developed, the kinetics of tumor formation were different, with a lag time of two to three weeks before tumor formation was noticeable (FIG. 6A, B). For 25% (n=3) of the mice tested, however, no difference was detectable in either the size of tumors or the kinetics of tumor development (FIG. 6D).

Similarly, in 70% (n=5) of the cases studied, tumor formation was reduced in DLD1 clones expressing caveolin-1 (FIGS. 7A, B, C). As for HT29-cav-1 clones, tumor formation was generally observed after an initial lag period of 2-3 weeks (FIGS. 7B, C). For 30% of the mice tested (n=2), however, no difference in the size of the tumor was detected (FIG. 7D).

### Example 6: Caveolin-1 Expression Levels in HT29-cav-1 and DLD1-cav-1 Cells Were Reduced Upon Tumor Formation in Nude Mice

The experiments with NIH-3T3 fibroblasts (FIG. 4) revealed that tumor formation resulted in cell populations with reduced caveolin-1 levels. Thus, possible explanations why tumor formation had occurred in some cases with transfected HT29 and DLD1 cells were that this process may either have led to elimination of caveolin-1 expression, despite being under the control of an exogenous promoter or to selection of cells with lower basal levels of caveolin-1 expression. To investigate these possibilities, cells were isolated from excised tumors, put back in culture and subsequently, after pure cell populations were available, examined for caveolin-1 protein expression (FIG. 8, ExTumor). Directly after plating, cells derived from tumors were a mixture of host cells (mainly fibroblasts) and tumor cells, but only tumor cells underwent rapid proliferation. By contrast host cells tended to detach and die rapidly. When culture plates were confluent after two passages, homogenous tumor cell populations, morphologically identical to parental HT29 or DLD1 cells, but with the additional ability to grow in the presence of hygromycin B, were obtained. In these cells, basal levels of caveolin-1 expression were reduced when compared to those observed for HT29-cav-1 cells before injection into mice (FIG. 8, ExTumor and BI respectively). Nevertheless, caveolin-1 expression could still be induced by the addition of IPTG. Thus, selection for HT29 cells expressing lower levels of caveolin-1 occurred upon tumor formation in nude mice. Similar results were obtained with DLD1 cells.

### Example 7: Selection for Methotrexate Resistance and Metastatic Potential Enhanced Caveolin-1 Expression in Colon Carcinoma Cells

The previous experiments strongly favored the notion that tumor formation in humans and in nude mice correlated with reduced caveolin-1 expression levels. Alternatively, it became of interest to examine whether low caveolin-1 expression levels were an irreversible state in colon carcinoma cells. Given that more differentiated cells tend to express higher caveolin-1 levels (Kandror, K. V., Stephens, J. M., and Pilch, P. F. Expression and compartmentalization of caveolin in adipose cells: coordinate regulation with and structural segregation from GLUT4, J. Cell Biol. 129.- 999-1006, 1995), culture conditions promoting cell differentiation may be expected to enhance caveolin-1 expression in colon carcinoma cells. Indeed, the methotrexate-resistant, more differentiated HT29 clones 5M12 and 5M21 (Lesuffleur, T., Barbat, A., Dussaulx, E., and Zweibaum, A. Growth adaptation to methotrexate of HT-29 human colon carcinoma cells is associated with their ability to differentiate into columnar absorptive and mucus-secreting cells, Cancer Res. 50: 6334-43, 1990), expressed significantly higher levels of caveolin-1 than parental HT29 cells, with the difference being greatest for the enterocytic clone 5M12 and less apparent for the mucous-secreting 5M21 cells (FIG. 9A). However, caveolin-1 was neither detectable in Caco2 cells cultured normally with frequent passaging (proliferative, undifferentiated cells; see FIG. 2) nor in cells left for 5 weeks without passaging (differentiated cells) (Rousset, M. The human colon carcinoma cell lines HT-29 and Caco-2: two in vitro models for the study of intestinal differentiation, Biochimie. 68 1035-40, 1986).

Taken together, these experiments suggest that variations in culture conditions favoring differentiation have little effect on caveolin-1 expression in colon carcinoma cells, but that phenotypic changes correlated with increased caveolin-1 expression when observed in conjunction with drug resistance.

To test the possibility that caveolin-1 expression might vary with metastatic potential, as suggested from experiments with human prostate cancer cells (Glenney, J. R., Jr. The sequence of human caveolin reveals identity with VIP21, a component of transport vesicles, FEBS Lett. 314. 45-8, 1992), two clones isolated from the colon carcinoma cell line Lovo (E2 and C5) that display higher metastatic potential than the parental Lovo cells were characterized (Remy, L., Lissitzky, J. C., Daemi, N., Jacquier, M. F., Bailly, M., Martin, P. M., Bignon, C., and Dore, J. F. Laminin expression by two clones isolated from the colon carcinoma cell line Lovo that differ in metastatic potential and basement-membrane organization, Int. J. Cancer. 51: 204-12, 1992). Indeed, caveolin-1 expression levels in the Lovo E2 and C5 were significantly higher than in the parental Lovo line (FIG. 9B), suggesting that up-regulation of caveolin-1 might Occur during metastasis.

### REFERENCES

1. Bishop, J. M. Molecular themes in oncogenesis, Cell. 64: 235-48, 1991.

2. Vogelstein, B., Fearon, E. R., Hamilton, S. R., Kern, S. E., Preisinger, A. C., Leppert, M., Nakamura, Y., White, R., Smits, A. M., and Bos, J. L. Genetic alterations during colorectal-tumor development, N. Engl. J. Med. 319: 525-32, 1988.

3. King, K. L. and Cidlowski, J. A. Cell cycle regulation and apoptosis, Annu. Rev. Physiol. 60:601-17, 1998.

4. Lengauer, C., Kinzler, K. W., and Vogelstein, B. Genetic instabilities in human cancers, Nature. 396: 643-9, 1998.

5. Rothberg, K. G., Heuser, J. E., Donzell, W. C., Ying, Y. S., Glenney, J. R., and Anderson, R. G. Caveolin, a protein component of caveolae membrane coats, Cell. 68: 673-82, 1992.

6. Glenney, J. R., Jr. and Soppet, D. Sequence and expression of caveolin, a protein component of caveolae plasma membrane domains phosphorylated on tyrosine in Rous sarcoma virus-transformed fibroblasts, Proc. Natl. Acad. Sci. USA. 89: 10517-21, 1992.

7. Koleske, A. J., Baltimore, D., and Lisanti, M. P. Reduction of caveolin and caveolae in oncogenically transformed cells, Proc. Natl. Acad. Sci. USA. 92: 1381-5, 1995.

8. Lee, S. W., Reimer, C. L., Oh, P., Campbell, D. B., and Schnitzer, J. E. Tumor cell growth inhibition by caveolin re-expression in human breast cancer cells, Oncogene. 16: 1391-7, 1998.

9. Racine, C., Belanger, M., Hirabayashi, H., Boucher, M., Chakir, J., and Couet, J. Reduction of caveolin I gene expression in lung carcinoma cell lines, Biochem. Biophys. Res. Commun. 255: 580-6, 1999.

10. Li, S., Couet, J., and Lisanti, M. P. Src tyrosine kinases, Galpha subunits, and H-Ras share a common membrane-anchored scaffolding protein, caveolin. Caveolin binding negatively regulates the auto-activation of Src tyrosine kinases, J. Biol. Chem. 271: 29182-90; 1996.

11. Li, S., Okamoto, T., Chun, M., Sargiacomo, M., Casanova, J. E., Hansen, S. H., Nishimoto, I., and Lisanti, M. P. Evidence for a regulated interaction between heterotrimeric G proteins and caveolin, J. Biol. Chem. 270: 15693-701, 1995.

12. Scherer, P. E., Okamoto, T., Chun, M., Nishimoto, I., Lodish, H. F., and Lisanti, M. P. Identification, sequence, and expression of caveolin-2 defines a caveolin gene family, Proc. Natl. Acad. Sci. USA. 93: 131-5, 1996.

13. Song, S. K., Li, S., Okamoto, T., Quilliam, L. A., Sargiacomo, M., and Lisanti, M. P. Co-purification and direct interaction of Ras with caveolin, an integral membrane protein of caveolae microdomains. Detergent-free purification of caveolae microdomains, J. Biol. Chem. 271: 9690-7, 1996.

14. Mineo, C., James, G. L., Smart, E. J., and Anderson, R. G. Localization of epidermal growth factor-stimulated Ras/Raf-1 interaction to caveolae membrane, J. Biol. Chem. 271:11930-5, 1996.

15. Li, S., Seitz, R., and Lisanti, M. P. Phosphorylation of caveolin by src tyrosine kinases. The alpha-isoform of caveolin is selectively phosphorylated by v-Src in vivo, J. Biol. Chem. 271: 3863-8, 1996.

16. Okamoto, T., Schiegel, A., Scherer, P. E., and Lisanti, M. P. Caveolins, a family of scaffolding proteins for organizing “preassembled signaling complexes” at the plasma membrane, J. Biol. Chem. 273: 5419-22, 1998.

17. Anderson, R. G. The caveolae membrane system, Annu. Rev. Biochem. 67: 199-225, 1998.

18. Engelman, J.A, Wykoff, C. C., Yasuhara, S., Song, K. S., Okamoto, T., and Lisanti, M.

P. Recombinant expression of caveolin-1 in oncogenically transformed cells abrogates anchorage-independent growth, J. Biol. Chem. 272: 16374-81, 1997.

19. Galbiati, F., Volonte, D., Engelman, J. A., Watanabe, G., Burk, R., Pestell, R. G., and Lisanti, M. P. Targeted downregulation of caveolin-1 is sufficient to drive cell transformation and hyperactivate the p⁴²/44 MAP kinase cascade, EMBO J. 17: 6633.-48, 1998.

20. Hurlstone, A. F., Reid, G., Reeves, J. R., Fraser, J., Strathdee, G., Rahilly, M., Parkinson, E. K., and Black, D. M. Analysis of the caveolin-1 gene at human chromosome 7q3 1.1 in primarytumours and tumour-derived cell lines, Oncogene. 18: 1881-90, 1999.

21. Lesuffleur, T., Barbat, A., Dussaulx, E., and Zweibaum, A. Growth adaptation to methotrexate of HT-29 human colon carcinoma cells is associated with their ability to differentiate into columnar absorptive and mucus-secreting cells, Cancer Res. 50: 6334-43, 1990.

22. Glenney, J. R., Jr. The sequence of human caveolin reveals identity with VIP21 a component of transport vesicles, FEBS Left. 314:45-8, 1992.

23. Peli, J., Schroter, M., Rudaz, C., Hahne, M., Meyer, C., Reichmann, E., and Tschopp, J. Oncogenic Ras inhibits Fas ligand-mediated apoptosis by downregulating the expression of Fas, EMBO J. 18: 1824-31, 1999.

24. Reymond, M. A., Sanchez, J. C., Schneider, C., Rohwer, P., Tortola, S., Hohenberger, W., Kirchner, T., Hochstrasser, D. F., and Kockerling, F. Specific sample preparation in colorectal cancer, Electrophoresis. 18: 622-4, 1997.

25. Reymond, M. A., Sanchez, J. C., Hughes, G. J., Gunter, K., Riese, J., Tortola, S., Peinado, M. A., Kirchner, T., Hohenberger, W., Hochstrasser, D. F., and Kockerling, F. Standardized characterization of gene expression in human colorectal epithelium by two-dimensional electrophoresis, Electrophoresis. 18:2842-8, 1997.

26. Plaetinck, G., Combe, M. C., Corthesy, P., Sperisen, P., Kanamori, H., Honjo, T., and Nabholz, M. Control of IL-2 receptor-alpha expression by IL-1, tumor necrosis factor, and IL-2. Complex regulation via elements in the 5′ flanking region, J. Immunol. 145: 3340-7, 1990.

27. Hunziker, W. and Meliman, I. Expression of macrophage-lymphocyte Fc receptors in Madin-Darby canine kidney cells: polarity and transcytosis differ for isoforms with or without coated pit localization domains, J. Cell Biol. 109: 3291-302, 1989.

28. Sambrook, J. F., Fritsch, E. F., and Maniatis, T. Molecular cloning: a laboratory manual. New York: Cold Spring Harbor Laboratory Press, 1989.

29. Melton, D. A., Krieg, P. A., Rebagliati, M. R., Maniatis, T., Zinn, K., and Green, M. R.

Efficient in vitro synthesis of biologically active RNA and RNA hybridization probes from plasmids containing a bacteriophage SP6 promoter, Nucleic Acids Res. 12: 7035-56, 1984.

30. Laemmli, U. K. Cleavage of structural proteins during the assembly of the head of bacteriophage T4, Nature. 227: 680-5, 1970.

31. Sager, R., Sheng, S., Anisowicz, A., Sotiropoulou, G., Zou, Z., Stenman, G., Swisshelm, K., Chen, Z., Hendrix, M. J., Pemberton, P., and et al. RNA genetics of breast cancer: maspin as paradigm, Cold Spring Harb. Symp. Quant. Biol. 59: 537-46, 1994.

32. Fra, A. M., Williamson, E., Simons, K., and Parton, R. G. Detergent-insoluble glycolipid microdomains in lymphocytes in the absence of caveolac, J. Biol. Chem. 269: 30745-8, 1994.

33. Kandror, K. V., Stephens, J. M., and Pilch, P. F. Expression and compartmentalization of caveolin in adipose cells: coordinate regulation with and structural segregation from GLUT4, J. Cell Biol. 129:999-1006, 1995.

34. Rousset, M. The human colon carcinoma cell lines HT-29 and Caco-2: two in vitro models for the study of intestinal differentiation, Biochimie. 68: 1035-40, 1986.

35. Remy, L., Lissitzky, J. C., Daemi, N., Jacquier, M. F., Bailly, M., Martin, P. M., Bignon, C., and Dore, J. F. Laminin expression by two clones isolated from the colon carcinoma cell line lovo that differ in metastatic potential and basement-membrane organization, Int. J. Cancer. 51: 204-12, 1992.

6. Engelman, J. A., Zhang, X. L., Galbiati, F., and Lisanti, M. P. Chromosomal localization, genomic organization, and developmental expression of the murine caveolin gene family (Cav-1, -2, and -3). Cav- 1 and Cav-2 genes map to a known tumor suppressor locus (6-A2/7q3 1), FEBS Lett. 429: 330-6, 1998.

37. Engelman, J. A., Zhang, X. L., and Lisanti, M. P. Sequence and detailed organization of the human caveolin-1 and -2 genes located near the D7S522 locus (7q31.1). Methylation of a CpG island in the 5′ promoter region of the caveolin-1 gene in human breast cancer cell lines, FEBS Lett. 448: 221-30, 1999.

38. Fra, A. M., Mastroianni, N., Mancini, M., Pasqualetto, E., and Sitia, R. Human caveolin-1 and caveolin-2 are closely linked genes colocalized with WI-5336 in a region of 7q31 frequently deleted in tumors, Genomics. 56: 355-6, 1999.

39. Yu, J., Zhang, L., Hwang, P. M., Rago, C., Kinzler, K. W., and Vogelstein, B. Identification and classification of p53-regulated genes, Proc. Natl. Acad. Sci. USA. 96: 14517-22, 1999.

40. Engelman, J. A., Zhang, X. L., and Lisanti, M. P. Sequence and detailed organization of the human caveolin-1 and -2 genes located near the D7S522 locus (7q31.1). Methylation of a CpG island in the 5′ promoter region of the caveolin-1 gene in human breast cancer cell lines, FEBS Lett. 448: 221-30, 1999.

41. Liu, J., Razani, B., Tang, S., Terman, B. I., Ware, J. A., and Lisanti, M. P. Angiogenesis activators and inhibitors differentially regulate caveolin- I expression and caveolae formation in vascular endothelial cells. Angiogenesis inhibitors block vascular endothelial growth factor- induced down-regulation of caveolin-1, J. Biol. Chem. 274: 15781-5, 1999.

42. Yang, G., Truong, L. O., Timme, T. I., Ren, C., Wheeler, T. M., Park, S. H., Nasu, Y., Bangma, C. H, Kattan, M. W., Scardino, P. T., and Thompson, T. C. Elevated expression of caveolin is associated with prostate and breast cancer, Clin. Cancer Res. 4: 1873-80, 1998.

43. Nasu, Y. Timrne, T. L., Yang, G., Bangma, C. H., Li, L., Ren, C., Park, S. H., DeLeon, M., Wang, J., and Thompson, T. C. Suppression of caveolin expression induces androgen sensitivity in metastatic androgen-insensitive mouse prostate cancer cells, Nat Med. 4:1062-4, 1998.

44. Lavie, Y., Fiucci, G., and Liscovitch, M. Up-regulation of caveolae and caveolar constituents in multidrug- resistant cancer cells, J. Biol. Chem. 273: 32380-3, 1998.

45. Cajot, J. F., Sordat, I, Silvestre, T., and Sordat, B. Differential display cloning identifies motility-related protein (MRP1/CD9) as highly expressed in primary compared to metastatic human colon carcinoma cells, Cancer Res. 57: 2593-7, 1997.

46. Garcia-Cardena, G., Fan, R., Stern, D. F., Liu, J., and Sessa, W. C. Endothelial nitric oxide synthase is regulated by tyrosine phosphorylation and interacts with caveolin-1, J. Biol. Chem. 271: 27237-40, 1996.

47. Ju, H., Zou, R., Venema, V. J., and Venema, R. C. Direct interaction of endothelial nitric-oxide synthase and caveolin-1 inhibits synthase activity, J. Biol. Chem. 272: 18522-5, 1997.

48. Ghosh, S., Gachhui, R., Crooks, C., Wu, C., Lisanti, M. P., and Stuehr, D. J. Interaction between caveolin-1 and the reductase domain of endothelial nitric-oxide synthase. Consequences for catalysis, J. Biol. Chem. 273:22267-71, 1998.

49. Michel, J. B., Feron, O., Sacks, D., and Michel, T. Reciprocal regulation of endothelial nitric-oxide synthase by Ca2+- calmodulin and caveolin, J. Biol. Chem. 272: 15583-6, 1997.

50. Engelman, J. A., Lee, R. J., Kamezis, A., Bearss, D. J., Webster, M., Siegel, P., Muller, W. J., Windle, J. J., Pestell, R. G., and Lisanti, M. P. Reciprocal regulation of neu tyrosine kinase activity and caveolin-1 protein expression in vitro and in vivo. Implications for human breast cance. J. Biol. Chem. 273:20448-55, 1998.

51. Couet, J., Sargiacomo, M., and Lisanti, M. P. Interaction of a receptor tyrosine kinase, EGF-R, with caveolins. Caveolin binding negatively regulates tyrosine and serine/threonine kinase activities, J. Biol. Chem. 272: 30429-38, 1997.

52. Oka, N., Yamamoto, M., Schwencke, C., Kawabe, J., Ebina, T., Ohno, S., Couet, J., Lisanti, M. P., and Ishikawa, Y. Caveolin interaction with protein kinase C. Isoenzyme-dependent regulation of kinase activity by the caveolin scaffolding domain peptide, J. Biol. Chem. 272: 3341 6-21, 1997.

53. Nystrom, F. H., Chen, H., Cong, L. N., Li, Y., and Quon, M. J. Caveolin-1 interacts with the insulin receptor and can differentially modulate insulin signaling in transfected Cos-7 cells and rat adipose cells, Mol. Endocrinol. 13: 2013-24, 1999.

54. Hulit, J., Bash, T., Ri, M., Galbiati, F., Albanese, C., Sage, D. R., Schlegel, A., Zhurinsky, J., Shtutman, M., Ben-Ze'ev, A., Lisanti, M. P., and Pestell, R. G. The cyclin D1 gene is transcriptionally repressed by caveolin-1, J. Biol. Chem., 2000.

55. Galbiati, F., Volonte, D., Brown, A. M., Weinstein, D. E., Ben-Ze'ev, A., Pestell, R. G., and Lisanti, M. P. Caveolin-1 expression inhibits Wnti beta-catenin/ Lef-1 signaling by recruiting beta-catenin to caveolae membrane domains, J. Biol. Chem., 2000.

56. Wary, K. K., Mariotti, A., Zurzolo, C., and Giancotti, F. G. A requirement for caveolin-1 and associated kinase Fyn in integrin signaling and anchorage-dependent cell growth, Cell. 94: 625-34, 1998.

57. Hermanek, P., Hutter, R. V. P., Sobin L. H., Wagner, G., and Wittekind, C. TNM-Atlas. Verlag, Heidelberg, New York: Springer, 1998.

We claim:

1. A method of treating a cell proliferation-associated disorder, comprising the step of administering a therapeutically effective amount of a caveolin polypeptide or a pharmaceutically acceptable salt thereof to a patient in need thereof.

2. The method of claim 1, wherein the caveolin is caveolin-1.

3. The method of claim 1, wherein the disorder is cancer.

4. The method of claim 3, wherein the cancer is non-steroid dependent carcinoma.

5. The method of claim 3, wherein the cancer is selected from the group comprising stomach carcinoma and colon carcinoma.

6. The method of claim 1, wherein the polypeptide is conjugated to a carrier.

7. The method of claim 6, wherein said carrier is selected from the group comprising an antibody, a liposome and an inert particle.

8. A method of treating a cell proliferation-associated disorder, comprising the step of administering a therapeutically effective amount of a caveolin nucleic acid to a patient in need thereof.

9. The method of claim 8, wherein the caveolin is caveolin-1.

10. The method of claim 8, wherein the disorder is cancer.

11. The method of claim 9, wherein the cancer is non-steroid dependent carcinoma.

12. The method of claim 9, wherein the cancer is selected from the group comprising stomach carcinoma and colon carcinoma.

13. The method of claim 8, wherein the nucleic acid comprises a vector.

14. The method of claim 13, wherein said vector is a viral expression vector.

15. A method for identifying a potential therapeutic agent for use in treatment of a pathology, wherein the pathology is related to aberrant expression or aberrant physiological interactions of a caveolin-1 polypeptide, the method comprising:
(a) providing a cell expressing the caveolin-1 polypeptide and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

16. The method of claim 15, wherein the pathology is cancer.

17. A method of identifying a carcinoma in a subject, the method comprising:
a) providing a test cell population from said subject, wherein at least one cell in said test cell population is capable of expressing a caveolin-1 nucleic acid;
b) measuring the expression of said caveolin-1 nucleic acid in said test cell population;
c) comparing the expression of said caveolin-1 nucleic acid to the expression of said caveolin-1 nucleic acid in a reference cell population comprising at least one cell whose carcinoma stage is known; and
d) identifying a difference in expression levels of the caveolin-1 nucleic acid, if present, in the test cell population and reference cell population, thereby identifying said carcinoma in said subject.

18. The method of claim 17, wherein said carcinoma is a non-steroid dependent carcinoma.

19. A method of assessing the efficacy of a treatment of a carcinoma in a subject, the method comprising:
a) providing a test cell population from said subject undergoing said treatment, wherein at least one cell in said test cell population is capable of expressing a caveolin-1 nucleic acid sequence;
b) detecting the expression of said nucleic acid sequence in said test cell population;
c) comparing the expression of said nucleic acid sequence to the expression of said nucleic acid sequence to a reference cell population comprising at least one cell whose carcinoma stage is known; and
d) identifying a difference in expression levels of the caveolin-1 sequence, if present, in the test cell population and reference cell population,
thereby assessing the efficacy of treatment of said carcinoma in said subject.

20. A method for identifying cancerous tissue, comprising:
a) contacting a test tissue comprising at least one cell at risk for or affected by cancer with an analyate capable of recognizing a caveolin-1 moiety;
b) quantifying binding of said analyate to said test tissue; and
c) comparing said binding of said analyate to said test tissue to binding of said analyate to a reference tissue comprising at least one cell whose carcinoma stage is known;
d) identifying a difference in binding levels of said analyate, if present, in the test tissue and reference tissue,
thereby identifying cancerous tissue.

21. The method of claim 20, wherein the analyate is an antibody and the caveolin moiety is a polypeptide.

22. The method of claim 20, wherein the analyate is a nucleic acid and the caveolin moiety is selected from the group consisting of genomic DNA, mRNA, and cDNA.

23. A composition comprising a caveolin-1 polypeptide and a pharmaceutically acceptable carrier.

24. An antibody capable of recognizing a caveolin-1 polypeptide.

25. A composition comprising a caveolin-1 nucleic acid and a pharmaceutically acceptable carrier.

26. The composition of claim 25, wherein said nucleic acid is a vector.
